(19) United States
(12) Patent Application Publication
(10) Pub. No.: US 2017/0000903 A1
(43) Pub. Date: Jan. 5, 2017

(54) CRYSTALLINE CELLULOSE GEL-BASED CRYPTANDS, SURFACE ACTIVE AGENTS, EMULSIONS AND VESICLES

(71) Applicant: UNIVERSITY OF SASKATCHEWAN, Saskatoon (CA)

(72) Inventors: Andrew A. OLKOWSKI, Saskatoon (CA); Bernard LAARVELD, Saskatoon (CA)

(73) Assignee: UNIVERSITY OF SASKATCHEWAN, Saskatoon, SK (CA)

(21) Appl. No.: 15/100,111

(22) PCT Filed: Nov. 27, 2014
(86) PCT No.: PCT/IB2014/066408

§ 371 Date: May 27, 2016

Related U.S. Application Data
(60) Provisional application No. 61/910,078, filed on Nov. 28, 2013.

(51) Int. Cl. A61K 47/48; A61K 47/38; A61K 47/46; A23L 33/10; C08J 3/03; C08L 91/00; A61K 31/4164; C09B 69/10; A61K 9/107; C08J 3/075

(52) U.S. Cl. CPC A61K 47/48961; A61K 9/107; A61K 47/38; A61K 47/46; C08J 3/075; C08J 3/03; C08L 91/00; A61K 31/4164; C09B 69/10; A23L 33/10; C08J 2401/02; C08J 2391/00; C08L 2201/52; C08L 2203/02; A23V 2002/00

(57) ABSTRACT

Crystalline cellulose gels useful in the stabilization of emulsions, including nanoemulsions, and for acting as cryptands or clathrates, and methods for their use and production are provided. In some embodiments, the emulsion is an oil-in-water emulsion. In some embodiments, such emulsions and/or vesicles produced in such emulsions, and/or the crystalline cellulose gel itself 15 are useful in the fields of drug delivery, pharmaceuticals, cosmetics, feed and food, paints and coatings, mining, or oil and gas recovery.

### REFERENCE TO RELATED APPLICATIONS

This application claims priority to, and the benefit of, U.S. provisional patent application No. 61/910,078 filed 28 Nov. 2013, the entirety of which is incorporated herein by reference.

### TECHNICAL FIELD

Some embodiments of the present invention pertain to carbohydrate-based surface active agents. Some embodiments of the present invention pertain to carbohydrate-based clathrates and cryptands useful for protecting or delivering a guest molecule. Some embodiments of the present invention pertain to carbohydrate-based emulsions. Some embodiments of the present invention pertain to vesicles formed using carbohydrate-based surface active agents and uses of same.

### BACKGROUND

Surface active compounds, commonly referred to as “surfactants”, are a group of amphiphilic organic compounds bearing inherent physico-chemical characteristics enabling them to lower the surface tension of a liquid or tension at the interface between diametrically different, normally immiscible, liquids.

In recent years, surfactants manufacturing has become increasingly dependent on petroleum, and concerns about environmental impact, as well as long-term sustainability, have led to a search for ecologically friendly, renewable bio-based products. Various bio-polymers may offer a very diverse source of starting materials for the development of bio-based surfactants.

Carbohydrate-based surfactants have been of interest for some time, due to their desirable performance properties and their potential to be derived from renewable biomass. Most carbohydrate-based surfactants utilize an O-glycoside linkage to glucose (or other sugar) molecule. More recently, a concept of carbohydrate C—C bond formation has been developed for the synthesis of new classes of carbohydrate-based surfactants.

Solid particles can also be used as emulsifying agents to produce so-called “Pickering emulsions”. Pickering emulsions are stabilized by suspended colloidal particles anchored at the oil-water interface. Such emulsions are described, for example, in US 2013/0122071.

Emulsions represent a class of two-phase systems of matter created from a mixture of two or more liquids that, under typical steady state conditions, are essentially immiscible. However, under some specific conditions, normally immiscible liquids can form a plethora of different types of emulsions. For example, even the simplest arrangement from the mixing of oil and water, two immiscible liquids, can result in at least two different states of emulsion; 1) oil-in-water emulsion, where the oil is the dispersed phase, and water is the dispersion medium, and 2) water-in-oil emulsion, where water is the dispersed phase and oil is the external phase (i.e. the dispersion medium). Multiple emulsions such as “water-in-oil-in-water” emulsions and “oil-in-water-in-oil” emulsions are also possible.

As a rule, emulsions do not form spontaneously, and energy input in the form of shaking, stirring, or homogenizing is needed to form an emulsion. Common emulsions are inherently unstable, and over time tend to revert to the stable state of the phases comprising the emulsion. An example of this is seen in the separation of the oil in various salad dressings where the emulsion tends to quickly separate unless shaken almost continuously.

Various surface active substances can increase the kinetic stability of emulsions so that the emulsion does not change significantly over time. The properties of surfactants such as wetting and penetration effect, emulsification, dispersion, foaming, and conditioning, are among the important and desirable effects of surfactants widely used across various industries.

Clathrates are chemical compounds consisting of a lattice of one type of molecule trapping and containing a second type of molecule. The name “clathrate complex” describes composites having a host molecule (cage) and a guest molecule (trapped within the cage of the host molecule by inter-molecular interaction). The term cryptand describes a compound (ligand) which binds substrates in a crypt, by interring the “guest molecule”, as in a burial. The original concept of “cryptant” describes molecules which are three dimensional analogues of crown ethers. It is desirable to provide compounds and compositions that can act as clathrates or cryptands.

Controlled drug delivery is a topic of considerable interest. There is interest in finding means to overcome the problems inherent in the use of many otherwise effective drugs which were abandoned in early investigations, or which are underused, because of poor bioavailability. Drug delivery using platforms such as solid lipid nanoparticles (SLN) or nanostructured lipid carriers (NLC) is an area of ongoing study.

Only a small proportion (less than ten percent) of new drug candidates show solubility and permeability properties requisite for practical pharmaceutical application. On the other hand, an estimated 25 to 40 percent of drug candidates fail to lead to successful drug products due to solubility limitations. Currently, some 30-40% of the drugs listed on the World Health Organization Essential Drug Inventory are inherently poorly water-soluble, and this quality hinders their full medicinal potential. Compared with highly soluble compounds, low drug solubility can manifest itself in a variety of undesirable outcomes such as low bioavailability and high inter-patient variability. It is desirable to develop compositions that can be used to assist in the delivery and/or administration of such drugs.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Some embodiments provide crystalline cellulose gels capable of acting as cryptands to form clathrates containing a guest molecule. In some embodiments, the cryptands or clathrates are used to deliver the guest molecule to a cell. In some embodiments, the crystalline cellulose moieties of the cryptand or clathrate are modified, for example by carboxylation, to provide an environment that is more receptive to, and therefore more likely to inter, a guest molecule. In some embodiments, the crystalline cellulose moieties of the cryptand or clathrate are modified, for example by attachment of suitable ligands or opsonins, to target delivery of the clathrate complex to specific cells.

Some embodiments provide crystalline cellulose gels that act as surface active agents to stabilize emulsions and form vesicles. In some embodiments, the emulsions are oil-in-water emulsions. In some embodiments, an aqueous phase, an oily phase, and a crystalline cellulose gel are combined together and mixed in any suitable manner to form an emulsion that is stabilized by the crystalline cellulose gel. In some embodiments, an active agent such as a drug or another biological molecule such as a hormone, nutrient, dye or nucleic acid is encapsulated in the dispersed phase of the emulsion, or inside vesicles formed in the emulsion. In some embodiments, the vesicles are readily taken up by cells, including white blood cells and red blood cells, allowing delivery of the drug or other biological molecule contained in the vesicle to a cell.

In some embodiments, the cellulose moieties of the crystalline cellulose gel that stabilizes the vesicles or emulsion is modified, for example by addition of appropriate ligands or opsonins, to facilitate targeted delivery of the drug or other biological molecule to specific cells. In some embodiments, the drug is used to treat cancer, and the crystalline cellulose gel is modified to target the vesicles to tumor cells. In some embodiments, the biological molecule is a nucleic acid such as DNA or RNA intended for gene therapy, and the crystalline cellulose gel is modified to target the vesicles to cells expressing proteins encoded by the DNA or RNA intended for gene therapy.

In some embodiments, vesicles or emulsions stabilized by crystalline cellulose gel are used to encapsulate a nutrient such as a vitamin or mineral, a functional food product such as a fatty acid, a hormone, or a prebiotic, to protect the encapsulated material, and optionally to facilitate targeted delivery of the encapsulated material to a specific region of the gastrointestinal tract of an animal.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows photographs of exemplary crystalline cellulose in powder form (panel (a)) and gel form (panel (b)) generated from flax shives using a catalytic reaction of biomass with an iron-based nanoparticulate catalyst.

FIG. 2 shows photographs of vials containing highly purified crystalline cellulose gel obtained from flax (left vial) and hemp (right vial).

FIG. 3 shows exemplary infra-red spectra of crystalline cellulose material isolated from flax processed using a catalytic reaction of biomass with an iron-based nanoparticulate catalyst to yield a crystalline cellulose gel superimposed with spectra from commercial microcrystalline cellulose.

FIG. 4 shows microscope images of nanocrystalline cellulose manufactured from flax shives using the catalytic process described in WO 2013/000074. Images show the crystal structures in colloidal suspension (panels (a) and (b)), which transform to a thin film form upon air drying (panel (c)). Images were digitally enlarged to cumulative magnification approximately 1200× from original magnification 400×.

FIG. 5A shows a photograph of a crystalline cellulose gel according to example embodiments at a concentration of approximately 2% (w/v). FIG. 5B shows a photograph of nanocrystalline cellulose obtained by drying crystalline cellulose gel after attempts to resuspend the nanocrystalline cellulose back into gel form were unsuccessful.

FIG. 6 shows images of the thixotropic physical characteristics of crystalline cellulose gel according to one example embodiment at a concentration of approximately 3% (w/v). Panels (a) and (c) show images of the crystalline cellulose gel after it has been left undisturbed for at least 90 minutes. Panels (b) and (d) show images of the crystalline cellulose gel after it has been agitated by vigorous shaking and assumes a liquid form.

FIG. 7 shows macro images of various exemplary emulsions obtained using crystalline cellulose gels obtained from flax biomass using the catalytic reaction described in WO 2013/000074 mixed with flax oil and water. Images were taken at time 0, 15 minutes, 30 minutes, 1 hour, 24 hours and 120 hours after agitation was stopped.

FIG. 8 shows the typical appearance of a crystalline cellulose gel based oil/water emulsion for one exemplary embodiment. The example embodiment contains crystalline cellulose gel obtained from flax shives (0.5% w/v) and flax oil (5% v/v), with the balance being de-ionised water.

FIG. 9 shows a microscopic image of the crystalline cellulose gel based oil/water emulsion shown in FIG. 8. The image was digitally enlarged to cumulative magnification approximately 1000× from the original magnification of 400×.

FIG. 10 shows examples of various multilamellar vesicles (indicated by arrows) observed in an exemplary crystalline cellulose gel based oil/water emulsion. The image was digitally enlarged to cumulative magnification approximately 1000× from the original magnification of 400×.

FIG. 11 shows the particle size distribution for the crystalline cellulose gel based oil/water emulsion shown in FIG. 8 as determined by dynamic light scattering.

FIG. 12 shows X-ray diffraction spectra of dried crystalline cellulose gel prepared from flax using the catalytic reaction described herein. The tracing denotes baseline corrected spectrum prior to de-convolution, and shaded tracings represent peaks identified using the minimum second derivative method.

FIGS. 13A, 13B and 13C show light microscope images of Clotrimazole mixed with water (FIG. 13A), dissolved in methanol (FIG. 13B), and first dissolved in methanol and then mixed with water (FIG. 13C).

FIGS. 14A, 14B and 14C show light microscopic images of native cellulose crystals found in crystalline cellulose gel in water (FIG. 14A), and the same mixed with Clotrimazole dissolved in methanol (FIGS. 14B and 14C).

FIG. 15 shows images of native cellulose crystals found in crystalline cellulose gel permeated with methylene blue (panel a), eosin (panel b), safranine (panel c), and brilliant green (panel d).

FIG. 16 shows photographs of test tubes containing crystalline cellulose gel treated with model dyes photographed at the start of the monitoring (top set) and at the completion of monitoring (bottom set). Dyes tested from left to right are safranine, hematoxilin, eosin, methylene blue, and congo red.

FIG. 17 shows the profile of methylene blue release from crystalline cellulose gel to the liquid phase.

FIG. 18 shows the profiles of malachite green, congo red, haematoxylin, and safranine release from crystalline cellulose gel to the liquid phase. The release profile of methylene blue is included for reference.

FIG. 19 shows profiles of eosin, light green, and brilliant green release from crystalline cellulose gel to liquid phase.

FIG. 20 shows infra-red spectra of dried crystalline cellulose gel prepared from flax processed using the described catalytic reaction (upper tracing, blue) and spectra from derivatized crystalline cellulose using above described process (lower tracing, red).

FIG. 21 shows photographs of examples of isolated white blood cells in control cultures (panel a) and cultures exposed to vesicles (panel b).

FIG. 22 shows photographs of examples of interactions of blood cells with vesicles. The specimen presented in panel a is focused to shows numerous blood cell containing vesicles (white arrows) and blood cells in the process of assimilation of vesicles (black arrow). The specimen presented in panel b is focused to show an example of large white blood cells loaded with vesicles (white arrow) and in the process of assimilation (black arrow).

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Previous work by the inventors on catalytic reactions for processing of various biomasses led to the discovery of novel methods for generation of crystalline cellulose, as described in PCT publication No. WO 2013/000074, which is incorporated by reference herein. Cellulose is a polysaccharide formed from a linear chain of several hundred to several thousand β(1-4)-linked D-glucose units. Hydroxyl groups on the glucose molecules from one chain can form hydrogen bonds with oxygen atoms on the same molecule or on a different molecule. The linear cellulose chains can engage in a high degree of three-dimensional bonding with each other to form a crystalline structure that is insoluble in water. Crystalline cellulose is to be distinguished from the amorphous fraction of cellulose found in biomass.

It has now been discovered that crystalline cellulose gel acts as a surface active agent and can stabilize emulsions. Traditional surfactants are typically small molecules that have a hydrophilic head and a hydrophobic tail. Cellulose is a larger molecule than traditional surfactants, and the discovery that crystalline cellulose gel can act as a surfactant is unexpected, and is distinct from the stabilization of emulsions by suspended colloidal particles of cellulose, as in the case of Pickering emulsions (for example as described in US 2013/0122071). The inventors have demonstrated that crystalline cellulose gel can be used to form emulsions with a high level of stability. In some embodiments, crystalline cellulose gel can be used to form vesicles having relatively uniform shape and dimensions, and/or to form multilamellar vesicles. In some embodiments, crystalline cellulose gel can be used to form nanoemulsions or nanoparticulate sized vesicles. In some embodiments, vesicles formed using a crystalline cellulose gel can be used to facilitate delivery of small molecules into cells. In some embodiments, vesicles formed using a crystalline cellulose gel can be used in any application in which vesicles and emulsions are currently used, including drug delivery and encapsulation and delivery of nutrients or food products, as well as various industrial applications.

Emulsions and vesicles formed with crystalline cellulose gel have potential applicability in a number of industries, including feed and food, pharmaceutical (including drug delivery, administration and dosing), cosmetic, paint and coating, oil and gas, mining, and any other industries where surfactants, emulsions or vesicles are currently employed.

Furthermore, it has now been discovered that crystalline cellulose gel itself has a liquid crystal structure, and can be used as a cryptand or clathrate, to surround a guest molecule and thereby facilitate delivery of that guest molecule to cells, including to a desired target cell or location.

Without being bound by theory, the crystalline cellulose gel produced according to some embodiments of the present invention is comprised of basic liquid crystal structures, wherein crystalline cellulose gel molecules have both hydrophobic and hydrophilic moieties. The fact that crystalline cellulose gel structures form vesicles with oil in an aqueous phase indicates that some portions of these structures are lipophilic (i.e. hydrophobic), because they interact with lipid molecules. In some embodiments, observation of vesicles produced from an oil-in-water emulsion stabilized with crystalline cellulose gel under a microscope indicates that individual vesicles have a strong tendency to repel each other. Without being bound by theory, it is believed that such behavior can be explained if the surface of the vesicles possesses the same charge.

The structure of cellulose is polymer chains of glucose units connected by a (3-bond. Each chain glucose molecule has 3 hydroxyl (OH) groups, and terminal glucose molecules have 4 hydroxyl (OH) groups on each end. If any of these OH groups are dissociated, the surface charge of the vesicle formed from an emulsion stabilized with crystalline cellulose gel is believed to be predominantly negative. Without being bound by theory, it is believed that the presence of such negative charges on the surface of vesicles prevents coalescence or aggregation of vesicles, which is believed to contribute to the stability of the resulting emulsion.

Further without being bound by theory, the belief that crystalline cellulose gel molecules have both hydrophobic and hydrophilic regions is consistent with the helical structures of crystalline cellulose. It is believed that the differences in some forms of crystalline cellulose molecular structure that allow the molecules to form a gel can be explained by factors such as cellulose chain packing, primary alcohol orientation, hydrogen bonding systems, and the shape of the glucose rings.

In some embodiments, crystalline cellulose gel is produced by breakdown of cellulose-containing biomass with reactive oxygen species according to the process described in WO 2013/000074, i.e. by obtaining an iron-based nanoparticulate catalyst by oxidizing an aqueous solution comprising reduced iron, and then reacting biomass that is a source of cellulose in an aqueous slurry with a pH less than 7 with the nanoparticulate catalyst and hydrogen peroxide, and then recovering a colloidal cellulose fraction from which crystalline cellulose gel is obtained by concentration. This method produces native crystalline cellulose (i.e. crystalline cellulose with no chemical modifications). If subjected to further drying, the crystalline cellulose gel so obtained will yield microcrystalline cellulose and/or nanocrystalline cellulose as a dry film.

As outlined for example in WO 2013/000074, microcrystalline cellulose and nanocrystalline cellulose can be produced by other methods. However, such methods do not produce a crystalline cellulose gel as described herein. Typical methods of production of microcrystalline cellulose and nanocrystalline cellulose include, for example, strong mineral acid digest of highly pure cellulose, for example using 64% sulfuric acid, followed by mechanical size reduction. Mechanical size-reduction processes that can be used following the acid digest include ultrasonic treatment, cryogenic crushing and grinding, and homogenization such as fluidization.

Microcrystalline cellulose or nanocrystalline cellulose produced by sulfuric acid digest may be chemically different from native microcrystalline or nanocrystalline cellulose, for example, by being highly sulfated. The presence of sulfate functional groups may render the microcrystalline cellulose or nanocrystalline cellulose unsuitable for some applications, for example, food and pharmaceutical applications, because the sulfate groups may be allergenic.

As outlined in WO 2013/000074, treatment of biomass using an iron-based nanoparticulate catalyst at acidic pH with hydrogen peroxide yields crystalline cellulose as a colloidal suspension after removal of any lignin and hemicellulose fractions from the solid cellulose fraction and resuspension of the solid cellulose fraction. In some embodiments, the colloidal suspension of crystalline cellulose obtained after catalytic treatment includes two fractions, a heavy fraction that precipitates under the influence of gravity and a light fraction that remains in suspension.

In some embodiments, crystalline cellulose gel is obtained by condensing the light fraction so obtained. Any suitable method may be used, for example, evaporation of solvent, precipitation, centrifugation at sufficiently high g forces, ultra-filtration and/or nano-filtration.

In some embodiments, crystalline cellulose gel is obtained by conducting mechanical size reduction of the heavy fraction so obtained. Mechanical size reduction can be conducted in any suitable manner, for example by sonication, cryogenic crushing and grinding, or homogenization such as fluidization. The resultant product may be further diluted or condensed as may be required for any given application.

In some embodiments, the crystalline cellulose gel obtained as described above possesses physical attributes of a liquid crystal, i.e. the gel has properties between those of a conventional liquid and a solid crystal. In some embodiments, the crystalline cellulose gel is birefringent. In some embodiments, the crystalline cellulose gel exhibits a high degree of opalescence. In some embodiments, the crystalline cellulose gel exhibits dichroism. Examples of the occurrence of crystalline cellulose in liquid crystal format are described, for example, in Godinho et al. 2010. “Self-winding of helices in plant tendrils and cellulose liquid crystal fibers”, Soft Matter, 6, 5965-5970, which is incorporated by reference herein. In some embodiments, without being bound by theory, the liquid crystal form of the crystalline cellulose gel is believed to contribute to the formation, size and behavior of vesicles formed in emulsions and stabilized with crystalline cellulose gel.

In some embodiments, the crystalline cellulose gel obtained as described above has attributes of a hydrogel. Without being bound by theory, this allows crystalline cellulose gel to be viewed as a three-dimensional network of cellulose crystals cross-linked with the water matrix. This network structure forming crystalline cellulose gel results from crosslinking cellulose polymer chains with water via physical, ionic and covalent interactions. In some embodiments, the crystalline cellulose gel may contain between 95 to 99% or even higher of water, and so water occupies the bulk of the crystalline cellulose gel space.

The internal spaces created in hydrogels by crosslinking with water can be used to accommodate various molecules, or even three-dimensional structures, because crystalline cellulose gel itself has three-dimensional structure. Because the cellulose moieties (which are constituents of the crystalline cellulose hydrogel) are by nature predominantly hydrophobic, but are forming hydrogel structures crosslinked by water, it is possible that in some embodiments, both hydrophobic and hydrophilic compounds (or structures) could be accommodated within the crystalline cellulose gel. The crystalline cellulose gel lattice behaves like a cryptand or clathrate, allowing the incorporation of guest molecules within the gel lattice. Because the crystalline cellulose gel is a liquid crystal and biological membranes also comprise a liquid crystal structure, it can be soundly predicted that the crystalline cellulose gel will facilitate delivery of the interred guest molecule into biological systems, including cells.

In some embodiments, the guest molecule interred within the crystalline cellulose gel is a biological molecule, such as a drug, hormone, nucleic acid (including DNA or RNA), dye, nutrient, probiotic, vitamin, mineral, carbohydrate, amino acid, or the like. In some embodiments, the biological molecule is a drug with poor solubility in water. In some embodiments, the drug is an aromatic compound. In some embodiments, the drug has a chemical structure that is a derivative of the imidazole ring. In some embodiments, the drug is an antifungal drug. In some embodiments, the biological molecule is a dye. In some embodiments, the dye is an aromatic compound. In some embodiments, the biological molecule is an aromatic compound, including a drug or a dye. In some embodiments, the biological molecule contains phenolic rings. In some embodiments, the biological molecule contains both phenolic rings and secondary, tertiary and/or quaternary nitrogen.

Cellulose is present in many different forms of biomass, and the crystalline cellulose gel according to some embodiments can be produced from any source of cellulose. In some embodiments, the crystalline cellulose gel is produced from agricultural biomasses or forestry by-products. In some embodiments, the crystalline cellulose gel is produced from plant biomasses or bacterial cellulose. In some embodiments, the crystalline cellulose gel is produced from cotton, cereal straw (including wheat, oat, barley or rice straw), grass straw, or from flax straw or hemp straw (including shives (hurd), bast fiber, or whole straws), which are all examples of agricultural biomasses. In some embodiments, the crystalline cellulose gel is produced from wood, which is an example of a forestry by-product. In some embodiments, the crystalline cellulose gel is produced from hemp straw, hemp bast fiber, hemp shives, flax straw, flax bast fiber and/or flax shives. Hemp and flax are examples of biomass that may be perceived by at least some members of the public to have environmental or health benefits as compared with other sources of biomass.

The inventors have now demonstrated that crystalline cellulose gels offer tremendous potential for the generation of surface active compounds and emulsions, including the production and stabilization of nanoemulsions. The examples discussed below demonstrate that crystalline cellulose gels allow formation of very stable emulsions and facilitate uptake of vesicles in such emulsions into cells. Emulsions and vesicles produced in such emulsions, including nanoparticle-sized vesicles, have a number of potential applications, including cosmetics, feed and food, drug delivery, pharmaceuticals, paints and coatings, mining, and oil and gas recovery.

In some embodiments, crystalline cellulose gels possess some highly desirable physico-chemical properties consistent with those typically attributed to surfactants, and allow formation of stable emulsions specifically well suited for biomedical applications. In some embodiments, crystalline cellulose gels allow formation and stabilization of nanoemulsions.

In some embodiments, an emulsion is formed using any desired relative amount of an oil-based component and an aqueous component to form an emulsion stabilized by a crystalline cellulose gel. In some embodiments, the emulsion is an oil-in-water emulsion. In some embodiments, an emulsion stabilized with a crystalline cellulose gel is provided with water/oil mixtures ranging from 0 to 50% by volume of oily base, or any value therebetween, e.g. 5%, 10%, 15%, 20%, 25%, 30%, 35%, 40%, or 45% of oily base.

In some embodiments, exemplary oily bases include a range of plant-based, animal-based or vegetable oils with desirable physico-chemical and/or pharmacological properties. In some embodiments, flax oil is used as the oily base. In the examples described herein, an exemplary flax oil base was selected for testing based on the known health benefits and pharmacological properties of flax oil. Other potentially suitable oily bases that are plant-based oils include hemp oil, canola oil, corn oil, sunflower oil, safflower oil, soy oil, coconut oil, palm oil, sesame oil, olive oil, or the like. In some embodiments, the oily base is an animal-based oil, for example, fish oil or milk fat. In some embodiments, the oily base is a mixture of two or more of the foregoing oils. Any desired oily base can be used in various embodiments, depending on the desired properties of the resultant emulsions or vesicles.

In some embodiments, a water-in-oil emulsion can be formed.

In some embodiments, the crystalline cellulose gel is present in the emulsion at a concentration between about 0.1% and 5% by weight or any value therebetween, e.g. 0.2%, 0.3%, 0.4%, 0.5%, 0.6%, 0.7%, 0.8%, 0.9%, 1.0%, 1.5%, 2.0%, 2.5%, 3.0%, 3.5%, 4.0% or 4.5%.

In some embodiments, the aqueous phase is water. In some embodiments, the aqueous phase is present in the emulsion at a concentration between about 45% and about 98% by volume, or any value therebetween, e.g. 50%, 55%, 60%, 65%, 70%, 75%, 80%, 85%, 90%, or 95%.

In some embodiments, the emulsion includes an active ingredient or other biologically relevant molecule. In some embodiments, the active ingredient or other biologically relevant molecule is encapsulated within the dispersed phase of the emulsion. In some embodiments, the active ingredient or other biologically relevant molecule is a drug, hormone, mineral, vitamin, nutrient, prebiotic, carbohydrate, amino acid, nucleic acid, dye, other bioactive agent or other molecule. In some embodiments, the active ingredient is a functional food ingredient, for example fatty acids such as omega-3 fatty acids, conjugated linoleic acid, semi-pure or pure anti-oxidants (which may be isolated from a natural source, for example, blueberries, grapes, pomegranates or the like), or the like. In some embodiments, the emulsion is used to deliver the active ingredient or other biologically relevant molecule to a target location. In some embodiments, the target location is a cell within the body of an animal. In some embodiments, the target location is a particular region of the gastrointestinal tract of an animal.

The emulsion can be formed in any suitable manner. In some embodiments, the emulsion is formed by combining an aqueous phase, an oily phase, and a crystalline cellulose gel to form a mixture. In some embodiments, an active ingredient or other biologically relevant molecule such as those listed in the preceding paragraph is added to the mixture. The mixture is then mixed to form an emulsion, for example by shaking, stirring, vortexing, homogenizing with a homogenizer, fluidization, cavitation, sonication, or any other method utilizing shear force, or the like.

In some embodiments, an emulsion is formed by combining an aqueous phase, an oily phase, and a crystalline cellulose gel to yield an oil-in-water emulsion. In some embodiments, the mixture is mixed by sonication to form an emulsion.

Based on the formation of stable emulsions and vesicles, including nano-emulsions and nanoparticle sized vesicles described in the Examples herein, and the demonstrated uptake of those vesicles by red and white blood cells, it can be soundly predicted that emulsions stabilized by crystalline cellulose gel in accordance with some embodiments of the present invention have potential utility in those fields where emulsions and/or vesicles stabilized by other surfactants are currently used. Such utility includes the potential to use emulsions and/or vesicles stabilized by crystalline cellulose gel in applications including pharmaceuticals and drug delivery, to deliver active ingredients or other biologically relevant molecules to cells.

In the food/feed industry, crystalline cellulose gel based emulsions according to some embodiments of the present invention may offer better means for the development of novel encapsulation technology for nutritional purposes. For example, vesicles formed with crystalline cellulose gel can be exploited for encapsulation of micronutrients such as minerals and vitamins, or other bioactive dietary ingredients in feed and food products, including nutritional supplements, prebiotics, carbohydrates, amino acids, hormones, or the like. Encapsulation could increase bio-availability of many nutrients and bioactive compounds which, because of their chemical nature, are poorly bio-available, by facilitating uptake within the gastrointestinal tract of an animal such as a mammal, including a human, and/or by providing targeted delivery of the encapsulated material to a specific region of the gastrointestinal tract. In some embodiments, crystalline cellulose gels could be used to form vesicles for delivery of nutrients and bioactive compounds. In some embodiments, crystalline cellulose gels could be used to form nanoemulsions to provide nanoparticle-sized vesicles for delivery of nutrients and bioactive compounds. For an explanation of drug delivery systems applied to dietary supplements and nutrients, see Shoji and Nakashima, “Neutraceutics and Delivery Systems”, J. Drug Targeting 12(6):385-391, 2004, which is hereby incorporated by reference.

For example, in one potential application in the food industry, encapsulation of nutrients or bioactive compounds in vesicles stabilized by crystalline cellulose gel could potentially be used to deliver encapsulated material through the rumen of a ruminant animal to the large or small intestine of that animal. In particular, the presence of bacteria within the rumen of a ruminant animal may make it difficult to provide nutrients (for example specific amino acids, vitamins or minerals) or other bioactive compounds (for example, prebiotics or carbohydrates) to the intestine of the animal. Crystalline cellulose is not digested by bacteria or by ruminants, and therefore vesicles stabilized by crystalline cellulose gel may pass through the rumen of the animal, but such vesicles could be taken up by absorptive cells in the ruminant animal's small or large intestine after passing through the rumen to thereby deliver the encapsulated material to the cells of the animals gastrointestinal tract.

In some exemplary embodiments having utility in the food industry, crystalline cellulose gel/water/oil based emulsions as described above can be formulated as suspensions, creams, pastes, or foams providing considerable versatility in various culinary applications. An advantage of crystalline cellulose gel based systems in the feed/food and pharmaceutical industries is the fact that cellulose itself is a component, offering zero calories, inert taste, and no significant changes to the texture or flavor of the final product. Accordingly, some embodiments offer potential application in various “calorie wise” products (i.e. low calorie food products) that are popular among consumers.

In exemplary bio-medical applications, for example, in pharmacological applications, crystalline cellulose gel based multilammelar vesicles (MLV) or unilamellar vesicles (ULV) systems according to exemplary embodiments of the present invention can be used for delivery of drugs or other bioactive agents and/or vaccines. In some embodiments, crystalline cellulose gel based nanoemulsions can be used for delivery of drugs or other bioactive agents and/or vaccines. Crystalline cellulose gel is biologically inert, so it is a carrier offering good bio-compatibility. Crystalline cellulose gel also exhibits a liquid crystal structure, and biological membranes are arranged in the form of a liquid crystal, and therefore the use of crystalline cellulose gel to form a vesicle may also facilitate uptake of that vesicle into a cell. Moreover, different embodiments of crystalline cellulose gel based emulsions may include practically any oil with desirable and customized physico-chemical and/or pharmacological properties for any given application. For example, in an exemplary embodiment described herein, an exemplary flax oil emulsion was tested because of the known health benefits and pharmacological properties of flax oil.

In some embodiments, multilamellar vesicles formed from a mixture of an oily phase, an aqueous phase and a crystalline cellulose gel can be used to encapsulate a drug or other bioactive compound and provide a time-delayed and/or sustained release of the drug or other bioactive compound. For example, as the outermost layer of the multilamellar vesicle is broken down, a portion of the drug or other bioactive compound will be released, together with further vesicles containing the drug or other bioactive compound. After a further period of time, the outermost layer of the further vesicles so released will be broken down, releasing a further portion of the drug or other bioactive compound, and any further vesicles encapsulated within the further vesicles. Sequential release of the drug or other bioactive compound from multiple layers of encapsulated vesicles within the multilamellar vesicle can provide gradual release of the drug or other bioactive compound over time. Encapsulation of multilamellar vesicles within multilamellar vesicles thus offers the possibility of providing an extended release formulation through the successive breakdown of the outermost layer of successively encapsulated vesicles over time. In some embodiments, such multilamellar vesicles are formulated together with appropriate pharmaceutical agents so as to be suitable for oral, topical (including in the form of a skin patch), parenteral, transmucosal, inhalation, injection or other form of delivery to the body of an animal, including a mammal and including a human.

In vivo, vesicles made using crystalline cellulose gels according to some embodiments can act as micro- or nano-sized delivery vessels by several potential mechanisms or modes of action, for example but without being bound by theory: by direct fusion of the vesicle with the cell lipid bi-layer; by pinocytosis, where the cell actively engulfs the vesicle; or, by phagocytosis where the cell internalizes the vesicle into a phagosome. The phagocytic cells (e.g. neutrophils) can carry a drug or other biological molecule encapsulated within the vesicles to a target tissue or cell.

The inventors have demonstrated that vesicles formed using crystalline cellulose gels are readily taken up by both white and red blood cells. In some embodiments, vesicles stabilized with crystalline cellulose gels are used to deliver encapsulated drugs or other bioactive agents directly to red or white blood cells. In some embodiments, phagocytic cells, e.g. neutrophils, are used to carry an encapsulated drug or other molecule to a specific target tissue affected by an inflammatory response, because the phagocytic cells can take up vesicles stabilized with crystalline cellulose gels containing the encapsulated drug or other molecule, and further are strongly attracted to tissues or organs affected by the inflammatory response via chemotaxis.

In some embodiments, vesicles stabilized with crystalline cellulose gels are used to deliver encapsulated anti-malaria drugs to red blood cells circulating within the bloodstream of a patient.

In some embodiments, rather than vesicles stabilized by crystalline cellulose gel being used as delivery vehicles, the crystalline cellulose gel itself is used as a cryptand or clathrate, to encase a guest molecule (for example, an active agent such as a drug, nutrient such as a mineral or vitamin, nucleic acid including DNA or RNA, hormone, dye, bioactive agent, prebiotic, carbohydrate, amino acid or other molecule). In some embodiments, crystals of the crystalline cellulose gel encasing an active ingredient are used to deliver the active ingredient to a target location. The use of crystalline cellulose gel itself as a cryptand or clathrate may be expected to be superior to the use of nanocrystalline cellulose as a particulate carrier (for example as described by Burt et al. in US 2014/0335132) because the crystalline cellulose gel itself exhibits a liquid crystal structure, and therefore can be expected to interact more strongly with and/or be more compatible with biological membranes, because biological membranes also have a liquid crystal structure.

In some embodiments, an active agent such as a drug or other biomolecule such as a nucleic acid, including deoxyribonucleic acid (DNA) or ribonucleic acid (RNA), can be encapsulated in a vesicle formed by preparing an emulsion using crystalline cellulose gel to deliver the active agent to cells in the body. In some embodiments, an active agent such as a drug can be encapsulated in a vesicle formed using an oily phase, an aqueous phase, and a crystalline cellulose gel under conditions such that the active agent is charged in solution. As the pH of the vesicle neutralizes, the charge on the active agent will also be neutralized, and the active agent may be able to pass through the cell membrane by diffusion.

In some embodiments, vesicles containing an active agent such as a drug or other molecule, or crystals of crystalline cellulose gel acting as a cryptand or clathrate to contain an active agent, may be targeted to specific cells by attaching appropriate molecules (e.g. ligands) to the glucose moieties of the crystalline cellulose gel to target the desired cells. In some embodiments, the crystalline cellulose gel based vesicles, or crystals of crystalline cellulose gel acting as a cryptand or clathrate, can be modified with ligands or opsonins that activate endocytosis in particular cell types to provide targeted delivery of the vesicles to those cell types. In some embodiments, the crystalline cellulose gel based vesicles are nanoparticles. Thus, some embodiments of the present invention provide new and alternative mechanisms for the delivery or controlled release of drugs or other biological molecules. The use of nanoparticles in drug delivery is described, for example, in the following publications:
Wacker M. 2013. Nanocarriers for intravenous injection—The long hard road to the market. Int J Pharm. 457: 50-62;
Hadinoto K, Sundaresan A, Cheow W S. 2013. Lipid-polymer hybrid nanoparticles as a new generation therapeutic delivery platform: A review. Eur J Pharm Biopharm. 85(3 PtA):427-43;
   each of which is incorporated by reference herein. In some embodiments, vesicles stabilized with crystalline cellulose gel, or crystalline cellulose gel acting directly as a cryptand or clathrate, may offer an improved composition for providing drug delivery and/or enhancing drug bioavailability as compared with solid lipid nanoparticles (SLN) or nanostructured lipid carriers (NLC).

In some embodiments, by manipulating the internal environment of the vesicle (for example by creating a proton gradient, for example by forming the vesicles under a first pH condition and transferring the vesicles to a second, different, pH condition), active substances may be transported in or out of cells. Via this mechanism, vesicles according to exemplary embodiments can potentially be used in bio-detoxification where the custom designed vesicles would act as sinks removing the toxin from the blood circulation, and safely disposing the toxin in organs such as the liver or kidney for bio-transformation or direct excretion in bile or urine. See for example Bertrand et al., (2010) “Transmembrane pH-Gradient Liposomes to Treat Cardiovascular Drug Intoxication” ACS Nano 4(12): 7552-8, which is incorporated by reference herein.

It is known in the art that the glucose molecules that make up cellulose can be readily chemically modified, for example through esterification, etherification, cationisation (e.g. by amidation), carboxylation, sulfonation, silylation, urethanization and/or polymer grafting. In some embodiments, chemical modification of the glucose molecules that make up the cellulose is used to provide desired properties of an emulsion or vesicle stabilized by the chemically modified crystalline cellulose gel, or of crystals of crystalline cellulose gel acting as a cryptand or clathrate. In some example embodiments, carboxylation of the glucose molecules that make up a crystalline cellulose gel is used to provide a more favourable environment for the encapsulation of positively charged, poorly water-soluble drugs in vesicles stabilized by crystalline cellulose gel, or in crystalline cellulose gel acting as a cryptand or clathrate.

Carboxylation of the glucose molecules that make up the crystalline cellulose gel can be carried out in any suitable manner. In some embodiments, carboxylation of the glucose molecules that make up the crystalline cellulose gel is carried out using performate. In some embodiments, the performate used in this reaction is prepared using glycerol as a catalyst, rather than using phenol as a catalyst as is known in the art. In one example embodiment, performate is prepared by combining hydrogen peroxide (35%) and formic acid (88%) at a volume ratio of between about 5:95 and 15:85 and adding glycerol (100%) as a catalyst at a volume ratio of between about 1:100 and 5:100 or 7.5:100. The reaction is allowed to proceed at room temperature for at least one hour to yield the desired performate reagent.

To carry out carboxylation of crystalline cellulose gel, crystalline cellulose gel is mixed with approximately 10% vol./vol. of the performate reagent, although any desired amount of performate reagent can be used. The reaction is incubated at room temperature for at least five hours, or overnight in some embodiments. After incubation, the carboxylated crystalline cellulose gel is separated from the supernatant in any suitable manner, for example filtration, decanting or the like, and can be washed with water to provide a neutral suspension.

In some embodiments, carboxylation of the crystalline cellulose gel using performate as described above is preferable to using existing techniques such as the use of persulfate (for example as described in WO 2011/072365) or TEMPO-mediated oxidation (2,2,6,6-tetramethylpiperidine-1-oxyl radical oxidation, e.g. as described in Nanoscale, 2011 3:71-85) because carboxylation using performate is more efficient and potentially less expensive.

In some embodiments, the ability to chemically modify the crystalline cellulose gel can be exploited to provide suitable binding sites for specific ligands such as opsonins, cell receptor active substances, monoclonal antibodies, or custom designed antigens and the like. The presence of such ligands would allow “arming” of vesicles stabilized by crystalline cellulose gel, or of crystalline cellulose gel acting as a cryptand or clathrate, with a tool that would seek out specific target tissues or cells and deliver an encapsulated active agent to that target.

In some embodiments useful in gene therapy, the use of crystalline cellulose gel-based vesicles may provide more efficient methods for transfection of DNA or other nucleic acid into a host cell than are currently available. The nucleic acid can be encapsulated within a crystalline cellulose gel-based vesicle formed from an emulsion as described herein to facilitate delivery of the nucleic acid to the host cell. In some embodiments, the nucleic acid is targeted to a particular type of host cell by arming the vesicles with a molecule that can seek out specific target tissues or cells as described above. In some example embodiments, white blood cells are targeted to take up vesicles containing DNA or other nucleic acid, and to deliver the DNA or other nucleic acid to the appropriate cells, for example by coupling an appropriate epitope onto the cellulose moieties of the crystalline cellulose gel.

In some embodiments useful in cancer therapy, the use of crystalline cellulose gel-based vesicles may provide a mechanism to deliver drugs or other bioactive molecules to tumor cells by modifying the cellulose moieties of the crystalline cellulose gel to target the vesicles to the tumor cells. In some example embodiments, white blood cells are targeted to take up vesicles containing drugs or other bioactive molecules, and to deliver the drugs or other bioactive molecules to the tumor cells by coupling an appropriate epitope onto the cellulose moieties of the crystalline cellulose gel.

In some exemplary embodiments for pharmacological applications, the delivery systems described herein can be readily incorporated into various formulations, emulsions, suspensions, creams, lotions, pastes, ointments or foams, or skin patches, for example using pharmaceutically acceptable carriers. Such compositions can be administered to an animal, including a mammal, including a human, in any suitable manner, for example, orally, topically (including by way of skin patch), parenterally, transmucosally, by inhalation, by injection, or the like.

In some embodiments, the crystalline cellulose gel based surfactants and emulsions are more stable and/or provide more robust performance than conventional surfactants or other carbohydrate surfactant-based products.

In some embodiments, by virtue of its polymeric and crystalline structure, the glucose moieties comprising crystalline cellulose gel offer tremendous structural flexibility that may be readily amenable to a wide range of customized chemical modifications by addition of various functional groups. Exemplary chemical modifications that can be carried out include carboxylation, sulfonation, esterification, etherification, silylation, urethanization, cationisation, including by amidation, polymer grafting, or the like. In some embodiments, non-covalent surface modifications of the crystalline cellulose gel can be made, for example via adsorption of surfactants, oppositely charged entities, or polyelectrolytes. Some potential modifications of cellulose are reviewed for example in Habibi, “Key Advances in the Chemical Modification of Nanocelluloses”, Chem. Soc. Rev. DOI: 10.1039/c3cs60204d, December 2013, which is incorporated by reference herein in its entirety.

In some embodiments, a product incorporating a crystalline cellulose gel can be produced from renewable biomass resources using minimal energy inputs. In some embodiments, generation of surfactants and emulsions using crystalline cellulose gel is conducted in accordance with the principles of “green chemistry” in a water-based, non-toxic environment.

In some embodiments, the process used to prepare the crystalline cellulose gel from natural biomass sources allows the generation of high purity material, essentially suitable for food and drug applications.

In one exemplary embodiment, vesicles formed in crystalline cellulose gel based emulsions including an aqueous phase and an oily phase have characteristics of clathrates or cryptands. In some such embodiments, the crystalline cellulose gel based emulsions are useful in drug delivery systems, cosmetics, pharmaceuticals, or the feed and food industries, or in the fields of paints and coatings, mining, and oil and gas recovery. In some embodiments, such crystalline cellulose gel based emulsions can be used to encapsulate a desired active agent to protect the active agent and/or to provide delivery of the encapsulated active agent to a predetermined target by attachment of an appropriate targeting moiety to the crystalline cellulose gel component of the emulsion.

Given the three-dimensional structure of crystalline cellulose gel-based nano- or micro-emulsions, in some embodiments, it may be possible to configure such emulsions to mimic cryptand-like behavior. In the context of the three-dimensional structure of the vesicle formed using crystalline cellulose gel (the host molecule), which is predominately hydrophobic but which is crosslinked with water, the resulting complexes may contain guest compounds (depending on the structure of the guest compound) that may be either hydrophilic or lipophilic. In some embodiments, the crystalline cellulose gel-based emulsions trapping hydrophilic guest molecules are water-in-oil emulsions. In some embodiments, the crystalline cellulose gel-based emulsions trapping lipophilic guest molecules are oil-in-water emulsions.

In some embodiments, it may be possible to manipulate the secondary structure of the cellulose molecules comprising the crystalline cellulose gel during the process of synthesis to provide a cryptand that would offer a versatile, custom designed physico-chemical environment. For example, in some embodiments, cross-linking crystalline cellulose gel glucose moieties with cations such as NH₄⁺ (amino) functional groups, or anions such as COO⁻ (carboxylic) functional groups would provide a three-dimensional interior environment consistent with a cryptand as a binding site (a nook) for “guest” ions (e.g. active agents such as pharmaceutical agents, nucleic acids, vitamins, minerals, dyes, and the like). Such an arrangement would be useful in industrial and pharmacological compound delivery applications, whether using crystalline cellulose gel directly as a cryptand or clathrate, or vesicles stabilized by crystalline cellulose gel.

In some embodiments, the crystalline cellulose gel has thixotropic properties. In some embodiments, the thixotropic properties of the crystalline cellulose gel may be exploited, for example to assist in the separation of target compounds or molecules from a mixture. In some embodiments, the crystalline cellulose gel may be agitated vigorously so that it behaves like a liquid, and then combined with a mixture comprising a target molecule. The target molecule may interact with and bind to the crystalline cellulose gel, which is then permitted to settle to a solid state. The crystalline cellulose gel containing the target molecule then settles from the mixture as a solid, and can be readily removed from the liquid mixture, for example by centrifugation, filtration, decanting or the like. In an alternative embodiment, the solid crystalline cellulose gel may be combined with a liquid mixture comprising a target molecule. The target molecule is permitted to bind to the crystalline cellulose gel in its solid form, and the solid fraction is removed from the mixture.

In some embodiments, after the solid crystalline cellulose gel combined with the target molecule has been removed from the liquid mixture, the crystalline cellulose gel can be agitated to return it to a liquid state, which may facilitate recovery of the target molecule from the crystalline cellulose gel.

In some embodiments where the thixotropic properties of crystalline cellulose gel are exploited, the target molecules are used in food applications, for example, vitamins, minerals, amino acids, carbohydrates, nutrients or other molecules relevant to nutrition. In some embodiments, the target molecules are chemical compounds used in industrial applications.

In exemplary embodiments suitable for application in the field of paints or coatings, an emulsion comprising an aqueous phase, an oily phase, and a crystalline cellulose gel can be prepared as described as above and used to encapsulate a dye or other protective coating and formulated for delivery to a surface such as metal, plastic, wood or the like.

In some embodiments, the process of producing emulsions using crystalline cellulose gels allows for complete control of the chemical environment of the process i.e. it can be chemically neutral, or can be directed towards basic or acidic environment, which provides versatility in formulations, which can be important for example in the context of medical applications such as drug delivery.

In some embodiments, the use of crystalline cellulose gel based micro- or nano-emulsions, where individual vesicles can act as clathrates or cryptands, or of crystalline cellulose gel that acts as a clathrate or cryptand directly, may facilitate a wide range of new strategies for use of crystalline cellulose gels in drug delivery systems.

In some embodiments having potential utility in the mining or oil and gas industries, crystalline cellulose gel may be used to stabilize emulsions or as a surfactant for use as a flocculant in various product recovery operations, wastewater treatment, remediation of petroleum contaminants in soil, and/or the manufacture of explosives for mining or the like.

In some embodiments, vesicles generated with crystalline cellulose gels are well suited for the task of nano- or micro-transporters for various biomedical applications.

### EXAMPLES

Some embodiments of the present invention are further described with reference to the following examples, which are meant to be illustrative and not restrictive in nature.

### Example 1.0

### Preparation of Crystalline Cellulose Gel from Flax Biomass

Crystalline cellulose gel was prepared using a method based on catalytic reactions using an iron-based nano-particulate catalyst as described in WO 2013/000074. Approximately 50 g of ground flax straw was dispersed in 1.8 L of water. This preparation was washed in reverse osmosis (RO) water, filtered through a colander, and re-suspended in 1.8 L of water. The consistency of this preparation was in the form of relatively dense slurry. The slurry was titrated with citrate reagent to obtain pH 3.8 and conditioned for 5 minutes. Following this, 15 ml of iron-based nano-particulate catalyst suspended in water was added, thoroughly mixed into the slurry mass, and pH was adjusted to 3.8. Following this, 15 ml of 35% hydrogen peroxide was added to obtain a relative REDOX potential (relative to water used in the medium) of approximately 140 to 180 mV. The catalytic reaction was carried out in a glass vessel on a thermostat controlled hot plate at 95° C. with constant agitation for approximately 16 to 24 hours.

Upon completion, the extracted cellulose fiber material (essentially consisting of pure cellulose fibers) was strained though a fine mesh colander, and rinsed 2 times with reverse osmosis (RO) water. The cellulose fiber then was subjected to a second catalytic reaction as described above in order to extract the crystalline form of cellulose. The process allows for controlled digestion of amorphous cellulose, and pure crystalline cellulose slurry is obtained upon completion of this process. The slurry is filtered through a paper filter (approximately 10 micron pore size). The residue of the crystalline cellulose retained on the filter is rinsed 2× on the filter, and under vacuum suction is dewatered to the point where a mass of wet cake consistency is formed. Throughout this process, complete hydration of the resulting cake is maintained at all times to prevent crystallization. The cake is then re-constituted with reverse osmosis (RO) water as required, and this preparation is subjected to sonication to convert the crystalline cellulose slurry to gel form.

The ratio of cellulose cake to water for sonication depends on the desired concentration of the resultant product. For example, a proportion of wet cake:water of approximately 1:10 v/v can yield crystalline cellulose gel with a consistency of approximately 3 to 4% w/v. In some cases, the actual yield may vary depending on the biomass from which crystalline cellulose was generated. For example, crystalline cellulose obtained from flax bast fiber will have lower density than that obtained from flax shives, and in general in most cases, crystalline cellulose obtained from flax will have higher density than crystalline cellulose obtained from hemp.

In this example, the sonication procedure is performed using an ultrasonic processor UIP 1000hd (Hielscher Ultrasonics GmbH, Germany) fitted with a BS2d34 sonotrode and B2-1.2 booster. In this procedure, the processor is set to operate at a frequency of 20 kHz, and ultrasonic treatment parameters used are: amplitude 100%, power output 60 to 70 W/cm², treatment time 15 minutes at a temperature of 70° C. After the completion of sonication, the resulting product assumes the consistency of crystalline cellulose gel.

As observed under light microscopy, the resultant gel laid as a thin film on a glass slide examined under dark field under 400× power appears as a fluid mass punctuated by several cellulose crystals, with variable sizes in the range of low micron or high nanometer. The product is a uniformly dispersed opalescent crystalline cellulose gel, which exhibits exceptionally uniform colloidal dispersion of the gel. Furthermore, the produced crystalline cellulose gels show a high degree of opalescent behavior, and this provides further evidence of dichroism. This feature is characteristically seen in highly dispersed systems, and the dichroism provides further evidence of the liquid crystal structure of the crystalline cellulose gel.

Example photographs of the powder form of nanocrystalline cellulose (panel (a)) and the gel form (panel (b)) of crystalline cellulose obtained through this process are presented in FIG. 1. The nanocrystalline cellulose shown in panel (a) was obtained by complete drying of the crystalline cellulose gel form shown in panel (b).

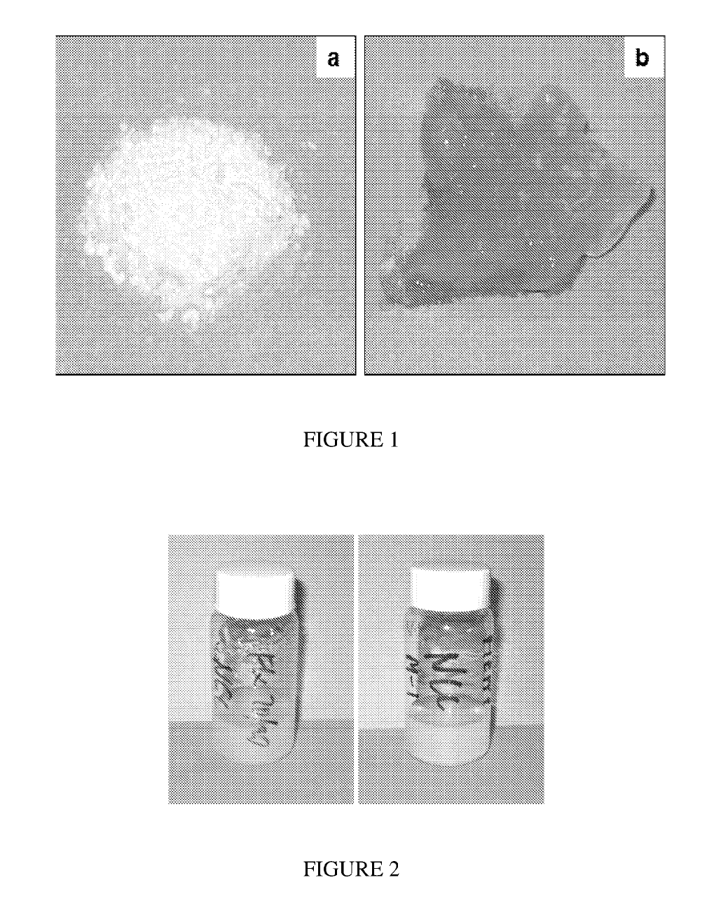

Example photographs of vials containing highly purified crystalline cellulose gel obtained from flax (left vial) and hemp (right vial) are shown in FIG. 2.

### Example 2.0

### FTIR Analysis of Nanocrystalline Cellulose

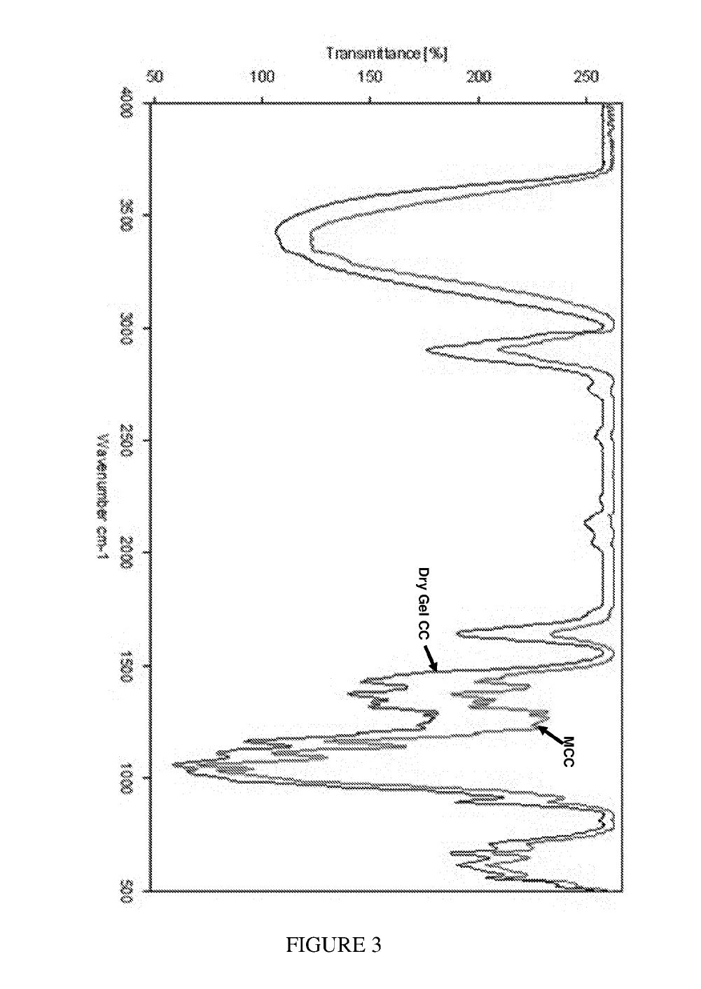

The obtained nanocrystalline cellulose fraction from Example 1.0 was further analysed using Fourier transform infrared spectroscopy (FTIR). The results are shown in FIG. 3. This analysis confirmed that spectra of nanocrystalline cellulose crystals obtained from flax in Example 1.0 are essentially identical to spectra obtained from commercial microcrystalline cellulose (MCC), as the spectra include substantially the same peaks, and do not include any additional peaks indicative of the presence of additional functional groups. In this context, the quantitative size of the peaks is not critical; the focus is on the qualitative similarity of the data (i.e. the pattern of deflections). Thus, FTIR spectra from dried crystalline cellulose gel are consistent with an international standard reference spectra (listed in Compendium of Food Additive Specifications. Addendum 5. FAO Food and Nutrition Paper—52 Add. 5, 2000). This example shows that the nanocrystalline cellulose crystals obtained from flax in Example 1.0 are native (i.e. chemically unmodified) cellulose.

The gel form of crystalline cellulose is not suitable for analysis by FTIR, and accordingly could not be subjected to the same analysis as the dried nanocrystalline cellulose. However, given that the spectra obtained for nanocrystalline cellulose obtained in Example 1.0 indicates that the nanocrystalline cellulose is native nanocrystalline cellulose, and given that the tested nanocrystalline cellulose is obtained by drying the crystalline cellulose gel obtained in Example 1.0, it is reasonable to conclude that the crystalline cellulose gel obtained in Example 1.0 comprises native cellulose (i.e. chemically unmodified cellulose).

FTIR imaging was performed using a Hyperion 3000 IR microscope coupled to a Tensor 27 interferometer (Bruker Optics, Billerica, Mass.). A KBr-supported Ge multilayer beamsplitter and a 64×64 pixel Focal Plane MCT detector (Santa Barbara Corp., Santa Barbara, Calif., USA) were used to measure spectra in the mid-infrared spectral region. Interferograms were recorded by scanning the moving mirror at 2.2 kHz, to an upper frequency limit of 3950 cm⁻¹ and with a spectral resolution of 4 cm⁻¹. 4×4 pixel binning was performed during acquisition. Single channel traces were obtained using the fast Fourier transform algorithm. Data analysis was performed using OPUS version 6.5 (Bruker Optics, Billerica, Mass., USA).

### Example 3.0

### Microscope Characterization of Nanocrystalline Cellulose

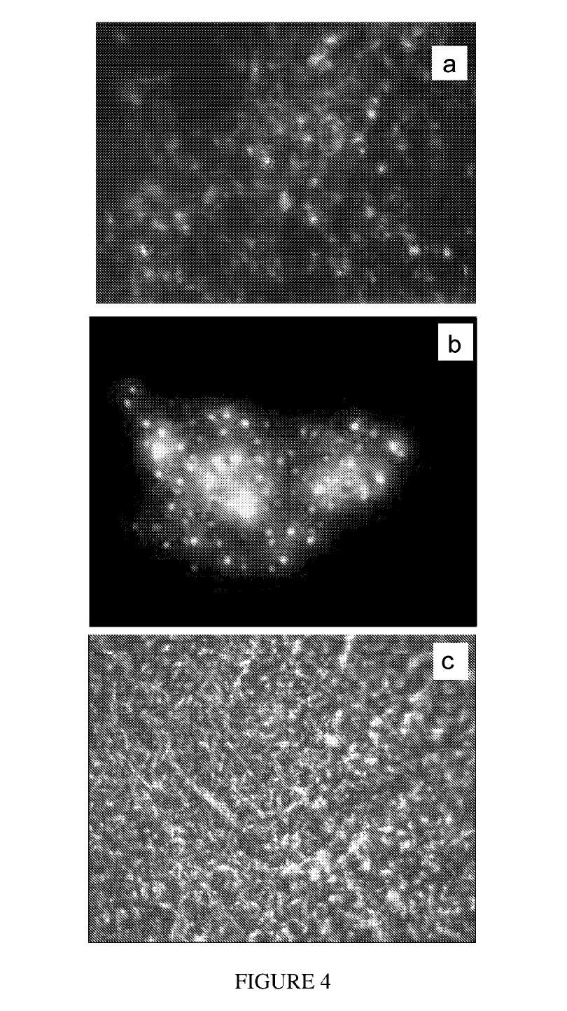

Nanocrystalline cellulose generated as in Example 1.0 analysed under the microscope shows morphology consistent with crystal structures in colloidal suspension, and forms a thin film in air dried form (FIG. 4, panels (a) and (b) show images of nanocrystalline cellulose in suspension, panel (c) shows the air dried form). Notably, this nano-polymeric material shows typical morphology consistent with crystal structures in colloidal suspension (panels (a) and (b)) which transform in a thin film form upon air drying (panel (c)).

Microscopic evaluation of crystalline cellulose gels under the dark field revealed the presence of highly dispersed dichroic material (FIG. 4, panels (a) and (b)). Noteworthy is the bulk of highly dispersed dichroic gel material which is punctuated by a small number of imbedded cellulose crystallites. This pattern of dispersion is interpreted as evidence of crystal fluidity consistent with a liquid crystal structure. The presence of cellulose in liquid crystal form is evidenced by dichroism, which characteristically occurs in liquid crystals due to the optical anisotropy. The presence of liquid crystal structures in crystalline cellulose gels is potentially advantageous because it is expected the products based on the crystalline cellulose gels would be highly compatible with biological systems, because biological membranes also are also arranged in a form of a liquid crystal.

### Example 4.0

### Physical Characterization of Nanocrystalline Cellulose

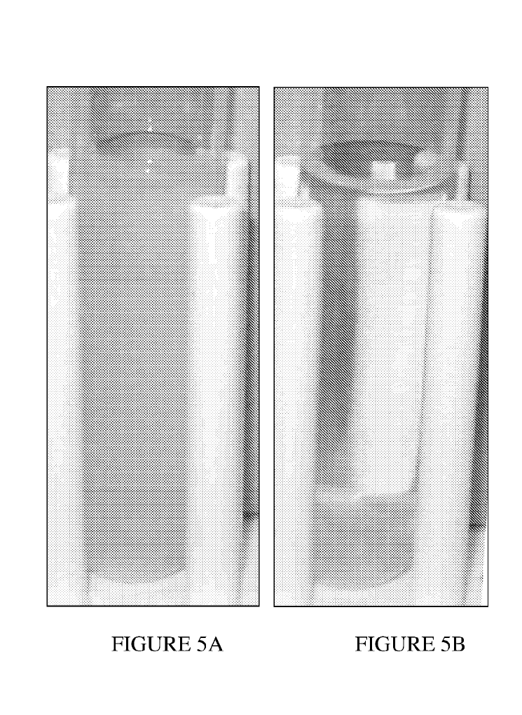

Efforts to form a crystalline cellulose gel using the dried nanocrystalline cellulose obtained above were unsuccessful. In particular, the thin film form illustrated in FIG. 4, panel (c) could not be redissolved into water, even after sitting for extended periods of time at room temperature. Virtually all attempts to return the dried form of nanocrystalline cellulose to gel form including shaking, vortex mixing, grinding and homogenization failed. Aggressive sonication for a long period did allow the conversion with less than about 10% yield of the dry nanocrystalline cellulose into the gel form. These results are shown in FIGS. 5A and 5B, where the native crystalline cellulose gel is observed to have a thick and uniform consistency (FIG. 5A), while dried nanocrystalline cellulose could not be resuspended in water to be returned back to the gel form and is observed as an insoluble mass (FIG. 5B).

This example demonstrates that crystalline cellulose gel as used in some embodiments of the present invention cannot simply be obtained by dissolving nanocrystalline cellulose in water.

### Example 4.1

### Thixotropic Physical Characteristics of Crystalline Cellulose Gel

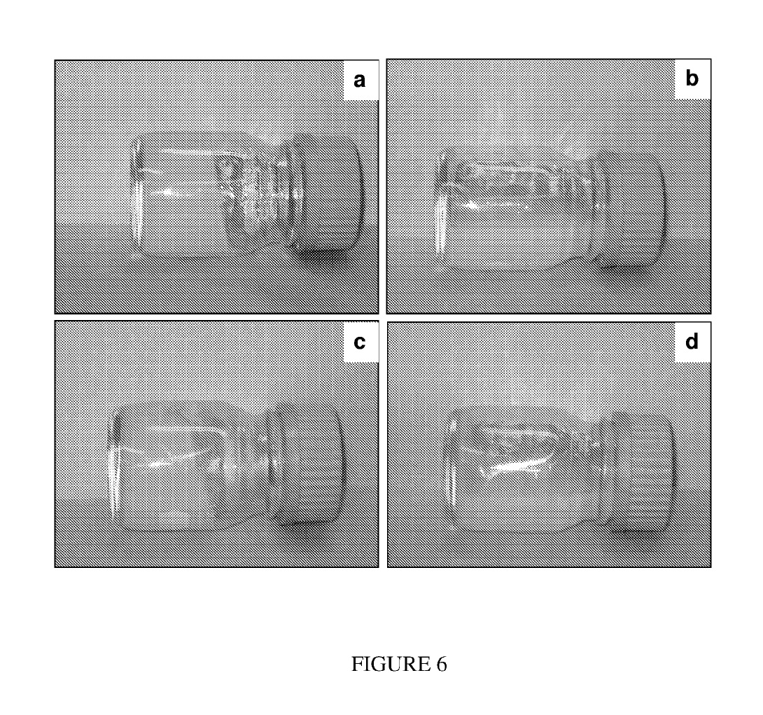

This example demonstrates that the crystalline cellulose gel in accordance with some embodiments of the present invention has thixotropic physical characteristics. FIG. 6 shows four images of a bottle containing crystalline cellulose gel at a concentration of approximately 3% (w/v). When left undisturbed, the crystalline cellulose gel in the bottle forms a thick semi-solid mass, which does not significantly change its shape even when the bottle is placed sideways (panel (a)). When the crystalline cellulose gel is agitated by vigorous shaking, it assumes a liquid form and behaves like a liquid (i.e. the crystalline cellulose gel flows horizontally when the bottle is placed sideways, as shown in panel (b)). When the liquid form of crystalline cellulose gel shown in panel (b) is allowed to stabilize undisturbed for about 90 minutes, the gel again assumes a semi-solid state (shown in panel (c)), which can be liquefied again upon agitation so that the crystalline cellulose gel again behaves like a liquid (panel (d)).

### Example 5.0

### Macro Scale Evaluation of Crystalline Cellulose Gel Based Emulsions

A series of experiments were conducted in order to evaluate the basic emulsifying potential of various crystalline cellulose gels obtained from flax straw or flax shives using an iron-based nano-particulate catalyst in acidic solution with hydrogen peroxide as described in WO 2013/000074. In this example, the biomass used was flax straw. Other sources of cellulose including flax shives and hemp were also assessed and similar results obtained.

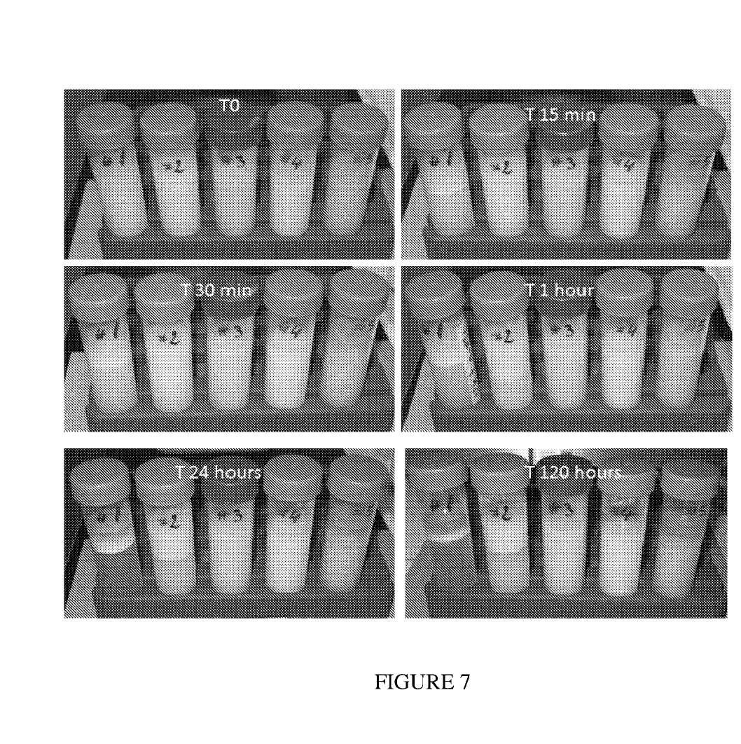

Initially, crystalline cellulose gels tested included various consistencies of gels ranging from 0.2% to 4% crystalline cellulose gel content in the emulsion by weight, and varied inclusion rates of flax oil, with the remainder of the mixture being water. Photographs of selected examples of emulsions based on crystalline cellulose gel/flax oil/water mixtures are presented in FIG. 7.

Samples for evaluation were prepared by mixing flax oil with water or water containing crystalline cellulose gel at the following rates: #1—20% (v/v) oil in water, no crystalline cellulose gel; #2—20% (v/v) oil in approximately 0.5% (w/v) crystalline cellulose gel; #3—20% (v/v) oil in approximately 2% (w/v) crystalline cellulose gel; #4 30% (v/v) oil in approximately 2% (w/v) crystalline cellulose gel; #5—50% (v/v) oil in approximately 2% (w/v) crystalline cellulose gel. Following vigorous agitation by shaking to form an emulsion, samples were set in the rack on bench top and all specimens were photographed immediately after shaking (TO). Samples were left undisturbed, and further pictures were taken at 15 minutes, 30 minutes, 1 hour, 24 hours, and 120 hours intervals.

Sample #1 (20% oil in water) showed clear separation of oil and water phase after 15 minutes, but none of the oil mixtures with crystalline cellulose gels separated up to 30 minutes. Although some separation of water and emulsion was apparent in tube #2 (20% oil in approximately 0.5% crystalline cellulose gel) after 1 hour, it is remarkable that emulsification appears to have stabilised in tube #2, and no further separation occurred. It is even more remarkable that mixtures containing approximately 2% crystalline cellulose gels (tubes #3, 4, and 5) did not show any evidence of phase separation during the 120 hours of initial monitoring. Further observations suggested that emulsions in all tubes appeared stable during the next 10 days of overt monitoring, and this suggests that their thermodynamic equilibrium has stabilised.

The results of this macro scale evaluation of various mixtures of base materials demonstrate the potential utility of crystalline cellulose gels in facilitating emulsification of essentially immiscible fluids such as water and oil. It is of particular interest to stress that two essentially immiscible compounds such as oil and water, mixed in presence of crystalline cellulose gel, even at very high oil:water ratios, form a very stable emulsion. Such features of these emulsions open new opportunities with potential applications across various industrial applications, including pharmaceuticals and drug delivery, foods, cosmetics, paints and coating, mining, and the oil recovery industry.

### Example 6.0

### Characterization of Stable, Crystalline Cellulose Gel Based Oil Emulsions

For the purpose of this study, crystalline cellulose gel was manufactured from flax shives using the catalytic process described in WO 2013/000074 in acidic solution with hydrogen peroxide using an iron-based nano-particulate catalyst using the protocol described for Example 1.0.

As described in WO 2013/000074, this catalytic processing results in the production of a solid cellulose fraction that can be separated from lignin and hemicellulose fractions, and then washed and harvested. The solid cellulose fraction can be resuspended in water to produce a colloidal suspension, having a colloidal light fraction and a heavier fraction that precipitates over time under the influence of gravity. The crystalline cellulose gel used in some embodiments of the present invention is harvested from the resulting colloidal light fraction. This fraction clearly separates from the heavy crystalline cellulose fraction produced by catalytic processing by gravity. In this example, microscopic analysis of the light fraction confirmed that the suspended colloid in the light fraction was comprised primarily of nanocrystalline cellulose. Particle size analysis showed typical bi-phasic particle distribution with one peak approximately in the 20 to 40 nm range, and the other peak in approximately the 800 nm range.

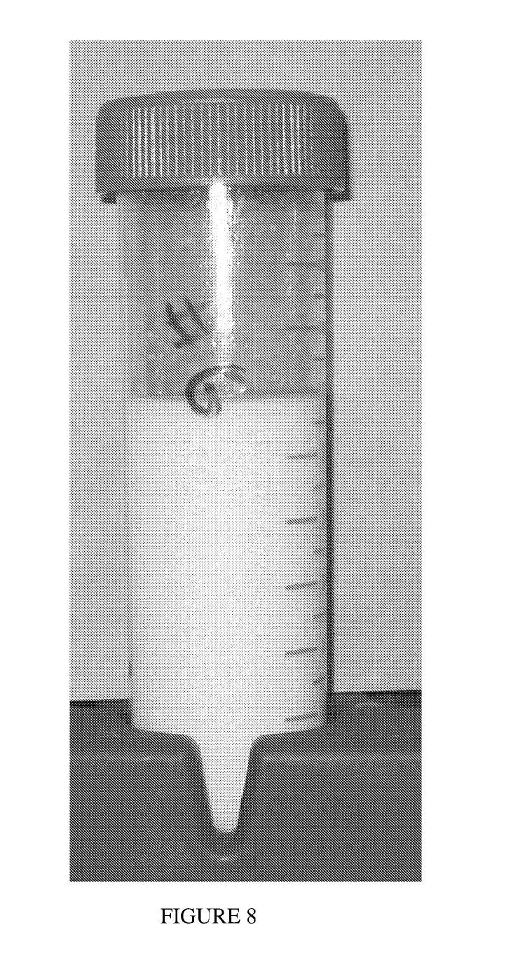

Approximately 0.5% w/v of crystalline cellulose gel in aqueous solution obtained from the light fraction after catalytic processing as described above were mixed with flax oil at various ratios. The mixture was sonicated for approximately 5 minutes. Following sonication, a stable saturated colloidal suspension (i.e. emulsion) was obtained. The final product containing crystalline cellulose gel obtained from flax shives (0.5% w/v) and flax oil (5% v/v), with the balance being de-ionised water, assumed the form of a white colored emulsion which is typical of water/oil based emulsions (FIG. 8). FIG. 8 shows highly stabilized emulsion which did not change after 72 hours. Further observation revealed that this emulsion remained stable for 7 days, and beyond.

### Example 6.1

### Microscopic Characterization of Crystalline Cellulose Gel Based Oil/Water Emulsions

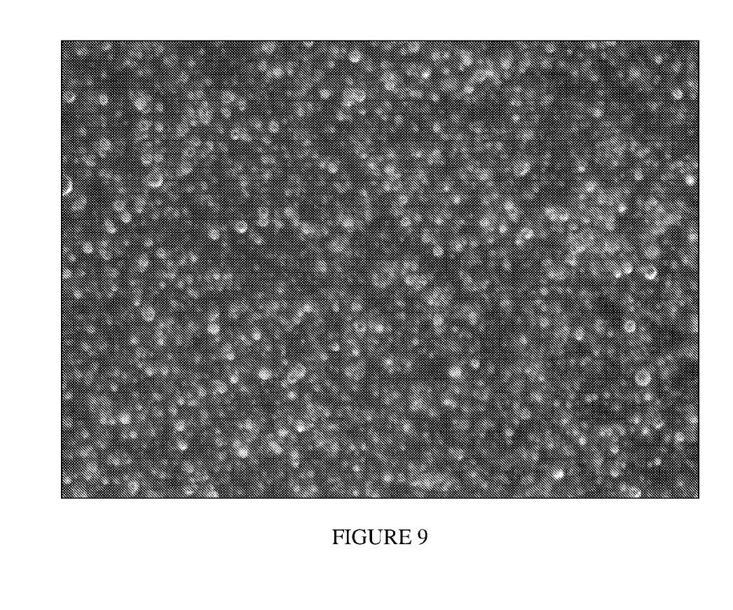

Microscopic examination of the emulsion product obtained in Example 6.0 revealed that the emulsion primarily consists of well defined, very regular, globe shaped vesicles (FIG. 9). The vesicles assumed a very symmetrical, orbicular structure. Noteworthy is the high degree of uniformity of the vesicle population and the fact that the vesicles remain well-separated (i.e. do not tend to aggregate). Without being bound by theory, it is believed that the slight negative charge of the cellulose in the crystalline cellulose gel tends to provide a slight repellent force that helps keep vesicles from aggregating.

Liposomes or lipid vesicles have been known since the 1960s. However, by comparing vesicle morphology seen in the exemplary product according to an embodiment of the present invention described above with the vesicles presented in some papers and patents, it was noted that vesicles generated with crystalline cellulose gel are considerably more uniform in comparison, and appear more robust as they appear to maintain very regular globular shape. Based on the results of this example that show the formation of stable and uniform vesicles within the emulsion, it can be predicted that vesicles formed as part of an emulsion stabilized by crystalline cellulose gel can be used to encapsulate oils and/or active agents for storage and/or targeted delivery in some embodiments of the present invention.

### Example 6.2

### Evaluation of Vesicle Morphology in Crystalline Cellulose Gel Based Oil in Water Emulsions

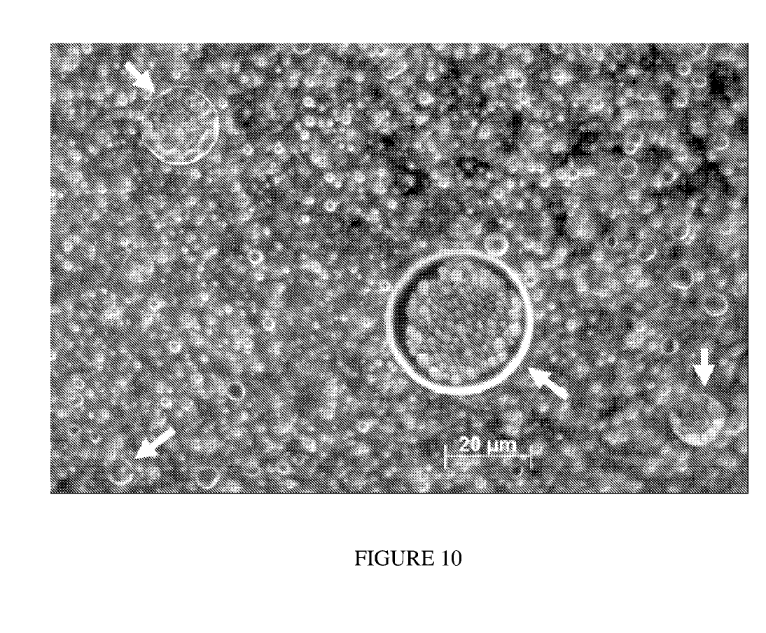

Further, microscopic examination of vesicle morphology present in various products prepared with crystalline cellulose gel based oil/water emulsions according to exemplary embodiments revealed the presence of larger structure that closely resembles the morphology of multilamellar vesicles (FIG. 10 as indicated with arrows). These multilamellar vesicles are comprised of many small vesicles arranged in the form of larger globular and very symmetrical structures of variable sizes (diameter).

Existing literature describes general types of vesicles as unilamellar vesicles (ULV) or multi lamellar vesicles (MLV) developed for a wide range of industrial applications. With the capacity to generate various classes of vesicles as demonstrated by this example, some embodiments of the present invention offer many useful alternatives in the transportation of both lipophilic and hydrophilic materials. Additionally, multi lamellar vesicles may be useful in producing time-release or gradual release formulation of drugs, and therefore have potential application in the medical field.

### Example 6.3

### Evaluation of Particle Size in Crystalline Cellulose Gel-Based Oil in Water Emulsions

An investigation of the particle size distribution of the crystalline cellulose gel stabilized oil/water emulsion shown in FIG. 8 was conducted by dynamic light scattering. The mean particle size was measured by dynamic light scattering spectroscopy using a Malvern Zetasizer, Nano ZS (Malvern Instruments Corp., Worcestershire, UK). Reverse osmosis (RO) water was used as a dispersant medium. Experiments were conducted at a temperature of 25.0° C. with a count rate of 247.5 kcps and a duration of 70 s. Results are presented in FIG. 11.

The Z-Average diameter of the particles measured was 364.1 nm. As shown in FIG. 11, a first peak in the particle size distribution was detected at 445.0 nm (78.6% intensity, standard deviation 138.9 nm) and a second peak in the particle size distribution was detected at 120.5 nm (21.4% intensity, standard deviation 30.0 nm). Thus, the oil/water emulsion obtained in Example 6.0 is a nano-emulsion containing nano-sized particles (i.e. vesicles).

Similar particle size distributions were measured for a sample of vesicles prepared from flax crystalline cellulose gel (approximately 1% w/v in reverse osmosis water) mixed with flax oil, which was added in excess in order to obtain an oil saturated mix. This preparation was sonicated for 60 minutes, and the mix was allowed to settle under gravity to separate excess oil from the saturated emulsion. Excess oil was decanted. The remaining emulsion preparation was enriched with several drops of cellulose gel to emulsify the remaining traces of oil, and the mixture was sonicated for 30 minutes. This approach allowed near-saturated crystalline cellulose-based emulsion to be produced, where there were no discernible amounts of unreacted free crystalline cellulose or unreacted free oil in the suspension. Visual evaluation revealed a very uniformly dispersed emulsion, which under microscopic examination showed that the vast majority of vesicles were in the sub-micron size range (i.e. were in the nano size range of a nano-emulsion).

The presence of a significant number of vesicles with a sub-micron particle size indicates that such emulsions are likely to be useful for applications like drug delivery or the targeted delivery of other molecules, where a small particle size is important to assist with uptake of the molecules encapsulated within such vesicles. Furthermore, because the vesicles are constructed with cellulose liquid crystals, it is reasonable to predict that such structure should more readily interact with biological membranes, which also have a liquid crystal structure. Therefore, one skilled in the art would reasonably predict that such vesicles will readily interact with biological systems and will facilitate delivery of molecules contained in such vesicles to biological systems.

### Example 7.0

### Evaluation of Crystallinity Indices of Crystalline Cellulose Gels

The relative content of crystalline cellulose is defined as the crystallinity index (CI). The crystallinity index (CI) provides information regarding the relative content of crystal lattice structure. Without being bound by theory, it is believed that the crystalline structure of the crystalline cellulose gel will cause its emulsions to behave like a cryptand or clathrate by interring a guest molecule. The name “clathrate complex” describes composites consisting of a host molecule (cage) and a guest molecule (trapped within the cage of the host molecule by inter-molecular interaction). The molecular characteristics of emulsions prepared using crystalline cellulose gel suggests that they provide a clathrate. The ability to act as a clathrate is important in certain applications, for example in pharmaceutical applications, including drug delivery applications, where a drug, dye or other molecule is to be delivered using a clathrate structure.

In this example, two dissimilar methods were used to measure the crystallinity index of crystalline cellulose gel: X-ray diffraction (XRD) and infrared spectroscopy (FTIR). The results show that the crystalline cellulose gel obtained as described herein has the attributes of a highly crystalline structure, as well as high purity. The use of two different methods to measure crystallinity index provides a high degree of confidence in the accuracy of the results.

### Example 7.1

### Evaluation of Crystallinity Index by Infrared Spectroscopy

Infrared spectroscopy was conducted as detailed for Example 2.0. The CI was calculated from second derivatives of FTIR spectra using the ratio of intensities at 1370/2900 cm⁻¹ wave number. Estimates of CI using FTIR method showed that the highly purified product contains 95+% crystalline cellulose.

### Example 7.2

### Evaluation of Crystallinity Index by X-Ray Diffraction

The X-ray diffraction analyses were performed using a model Empyrean, PANalytical, BV Lelyweg 1, 7602 EA (Almelo, The Netherlands). The instrument was operated with the following settings: Anode Material:Co, K-Alpha1 [Å]:1.78901, K-Alpha2 [Å]:1.79290, K-Beta [Å]:1.62083, K-A2/K-A1 Ratio:0.50000, generator 45 mA, 40 Kv. Goniometer Radius [mm]:240.00, Dist. Focus-Diverg. Slit [mm]:100.00. Scan Axis: Gonio, Start Position [°2Th.]:7.0084, End Position [°2Th.]:79.9724, Step Size [°2Th.]:0.0170; Scan Step Time [s]:101.6000, Scan was continuous; PSDLength [°2Th.]:2.12, Offset [°2Th.]:0.0000; Divergence slit was fixed, Slit Size [°]:0.5000, Specimen Length [mm]:10.00; Measurement Temperature [° C.]:25.00.

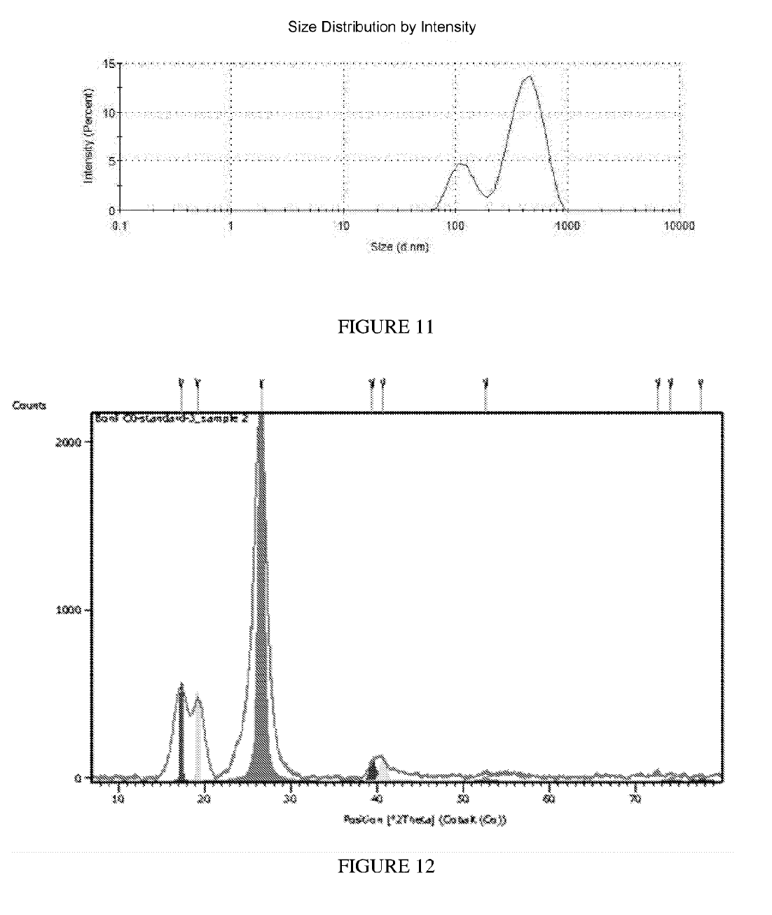

Estimated values of crystallinity from X-ray diffraction analysis showed crystallinity indices ranging from approximately 87 to 95% when evaluation was based on the X-ray diffraction peak height method or deconvolution method. An example of X-ray diffraction spectra for an exemplary crystalline cellulose gel product is presented in FIG. 12. It is noteworthy that based on the X-ray diffraction spectra, crystalline cellulose gels showed routinely at least 3 crystalline peaks, and in some gels up to five crystalline structure peaks were identified, as shown in the results presented in FIG. 12.

### Example 8.0

### Evaluation of Interaction of Crystalline Cellulose Gels with Other Compounds

Given the demonstration that crystalline cellulose gel has very high content of crystalline structures, the ability of the crystalline cellulose gels to interact with other compounds was evaluated to confirm that the crystalline cellulose gels themselves are well suited to act as cryptands or clathrates. The results demonstrate that not only do vesicles stabilized with crystalline cellulose gels act as cryptands or clathrates, but the crystalline cellulose gel itself can also act as a cryptand or clathrate to facilitate targeted delivery of interred guest molecules.

### Example 8.1

### Evaluation of Interaction of Crystalline Cellulose with Clotrimazole

Because of analytical limitations, in its native liquid form, it is difficult to visually evaluate the clathrate interaction of crystalline cellulose gel with other molecules. However, a model was developed whereby solid crystals of low micron size range inherently present in the crystalline cellulose gel were used to examine interactions of the crystalline cellulose gel with various drugs or dyes.

As a model compound for this study, the antifungal drug Clotrimazole was selected, which has the chemical structure (1) shown below. Clotrimazole is a colourless, crystalline, weakly alkaline substance, melting point 141°-145° C., soluble in acetone, chloroform, methanol and ethanol, and practically insoluble in water. This compound was chosen because of its physico-chemical properties such as insolubility in water and crystal structure. This drug shows poor oral absorption. Clotrimazole is an antifungal drug, which is a derivative of the imidazole ring, which is known to inhibit bio-transformation by cyt P450 enzymes. Other water-insoluble crystalline drugs or drugs with an aromatic structure would be expected to interact similarly with crystalline cellulose gel.

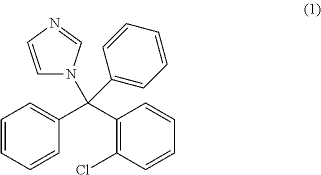 [SMILES: Clc1ccccc1C(c1ccccc1)(c1ccccc1)n1ccnc1]

To test the interaction of Clotrimazole with cellulose crystals, the drug was first mixed with water or with methanol at a rate of 1 mg per ml. The methanol solution was then mixed with 1% cellulose gel (treatment) and water (positive control). All samples were examined under a dark field microscope.

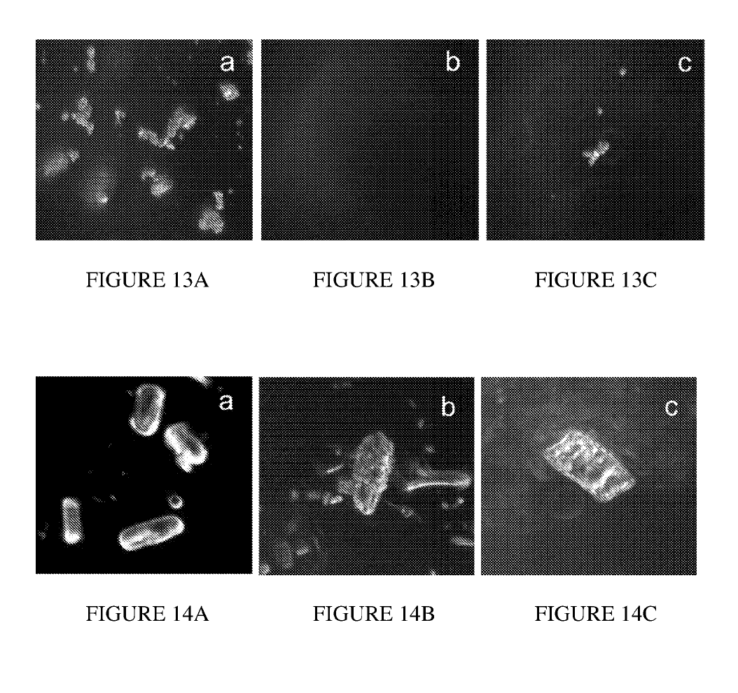

As evidenced in FIGS. 13A, 13B and 13C, Clotrimazole was not soluble in water, as this compound precipitated in the form of fine crystals (FIG. 13A). On the other hand, there were no precipitated crystals when Clotrimazole was mixed with methanol (FIG. 13B), and this indicates that this compound is totally dissolved in methanol. However, when the methanol solution of Clotrimazole was mixed with water, fine crystals precipitated (FIG. 13C). This test allowed observation of the changes of physico-chemical properties of Clotrimazole when a methanol solution of this compound was placed in a predominantly aqueous environment, as similar behaviour was expected when a methanol solution of Clotrimazole was mixed with crystalline cellulose gel, i.e. placed in a 99+% aqueous environment.

As predicted, when the Clotrimazole-methanol solution was mixed with an aqueous cellulose gel solution containing small solid cellulose crystals, fine Clotrimazole crystals were formed. Interestingly however, some of the precipitated drug was found imbedded in the crystalline cellulose matrix (FIGS. 14A, 14B and 14C). Notably, cellulose crystals resulting from interaction of cellulose with Clotrimazole solution shown in FIGS. 14B and 14C have different structures than the pure native cellulose crystals shown in FIG. 14A. It is apparent that in contrast to native cellulose crystals, which have more homogeneous crystal structure (FIG. 14A), when crystalline cellulose gel interacted with Clotrimazole, the resulting crystal structures have clearly heterogeneous crystal structure (FIGS. 14B and 14C). Therefore based on this analysis, it is reasonable to infer that when Clotrimazole dissolved in methanol is re-crystallized in predominantly aqueous environment upon mixing with cellulose gel (99+% water), quantities of Clotrimazole crystallites become incorporated in the lattice matrix of cellulose crystals.

### Example 8.2

### Interaction of Crystalline Cellulose Gel with Various Dyes

A set of experiments was conducted in which a set of dye compounds was used to visually evaluate interactions between crystalline cellulose gel and various chemical structures. A broad diversity of chemical structures were selected for testing, and considerations in compound selection also included diverse ionic charge and polarity ranging from strongly positively charged (predominantly cationic) compounds, neutral compounds, and strongly negatively charged (predominantly anionic) compounds. Many of these compounds contain phenolic rings and secondary, tertiary and/or quaternary nitrogen, which are also building blocks of many drugs, including Clotrimazole. A list of model dye compounds and absorbance maxima used for their spectrophotometric monitoring is provided in Table 1, and their structures are set forth below.

TABLE 1
Model dye compounds and their absorbance maxima.

| | Absorbance Maxima | |
|---|---|---|
| Tested Dye Compound | (nm) | Chemical Structure |
| Methylene Blue | 660 | (2) |
| Hematoxilin | 560 | (3) |
| Congo Red | 500 | (4) |
| Safranine | 520 | (5) |
| Malachite Green | 620 | (6) |
| Brilliant Green | 630 | (7) |
| Light Green | 630 | (8) |
| Eosin | 510 | (9) |

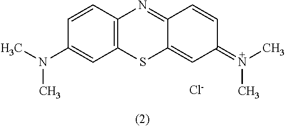

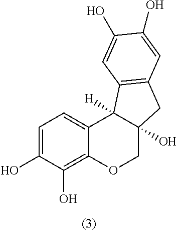 [SMILES: Oc1cc2c(cc1O)[C@@H]1c3ccc(O)c(O)c3OC[C@]1(O)C2]

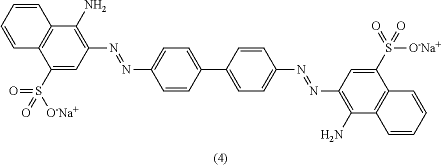

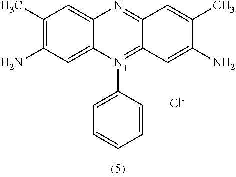

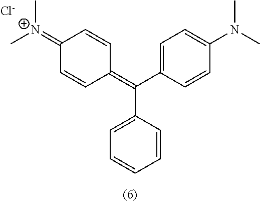

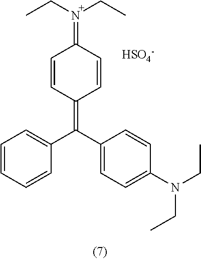

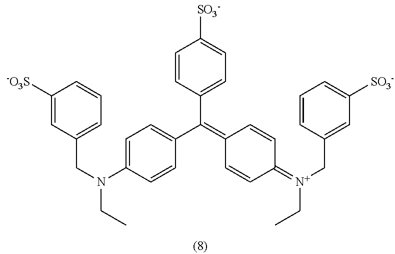

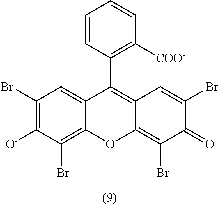

Approximately 2 ml aliquots of 3% w/v crystalline cellulose gel samples containing small solid cellulose crystals were thoroughly mixed with respective model dye solutions and incubated for 30 minutes at room temperature. The stained samples where then centrifuged. The supernatant was decanted, and the pellets containing crystalline cellulose were washed several times with reverse osmosis water and centrifuged. The cycles of washing and centrifugation were repeated until water in which cellulose gel was suspended was clear. After the final wash, small samples of each gel were taken for microscopic examination, and the gels in each tube were covered with a 10 ml column of RO water. These preparations served as media for monitoring the rate of dye release from crystalline cellulose gel to the surrounding liquid phase. Following a few minutes of sample equilibration, 500 μl of the liquid phase was withdrawn from each preparation for spectroscopic measurements to establish absorbance values at time 0. Subsequently, 500 μl samples of the liquid phase were collected at 2, 6, 12, 24, 48, and 72 hour intervals for monitoring of dye release to the liquid phase from the cellulose matrix over time.

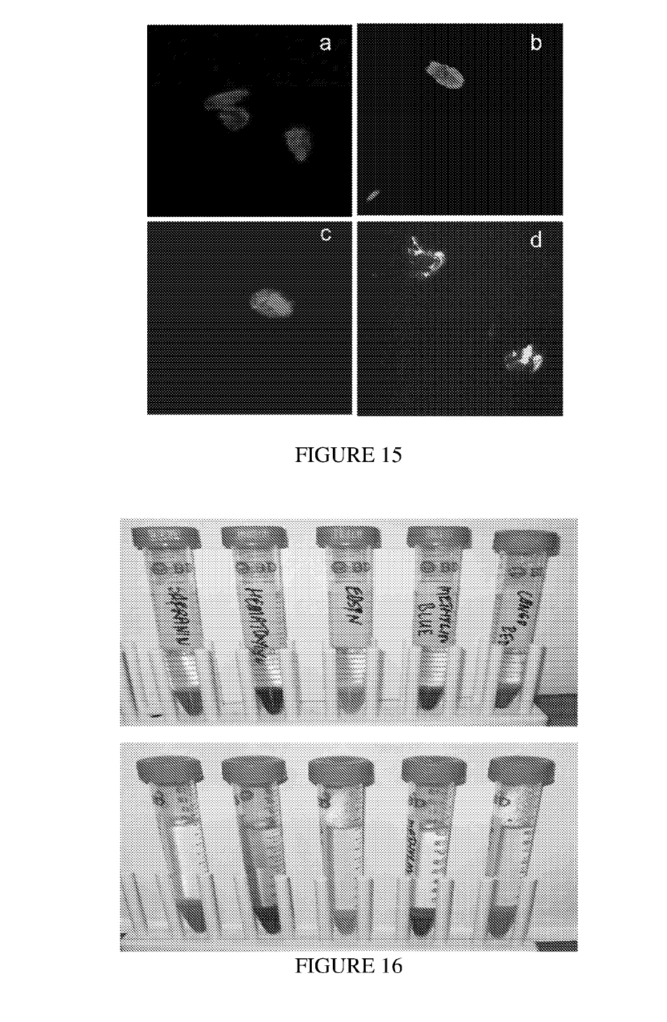

Strong interactions of the tested dye compounds with the crystalline cellulose gel was confirmed by examination of cellulose crystals contained within the gel using fluorescence microscopy. Examples of strong interactions between dye and cellulose crystals are presented in FIG. 15. Imaging with methylene blue (panel a) shows a strong blue colour associated with the cellulose crystals. Imaging with eosin (panel b) shows a strong red colour associated with the cellulose crystals. Imaging with safranine (panel c) shows a strong red color associated with the cellulose crystals. Imaging with brilliant green (panel d) shows a strong green color associated with the cellulose crystals.

FIG. 16 shows photographs demonstrating the release of dye over time from native cellulose crystals. The top set of images shows the test tubes at the start of monitoring, and the bottom set of images shows the test tubes after 72 hours of incubation at room temperature (i.e. approximately 21-22° C.). Dyes tested from left to right are safranine, hematoxilin, eosin, methylene blue, and congo red. In FIG. 16, at the start of incubation (top images) from left to right, the test tubes contain respectively red, purple, orange, blue and red precipitates, with a colourless solution above the precipitate. After incubation for 72 hours, the coloured dye has diffused out of the precipitated cellulose crystals in tubes 2, 3 and 5 from the left (hematoxilin, eosin and congo red), so that in these tubes there is respectively a strongly purple, orange and orange-coloured solution above the precipitate. In tube 1 from the left (safranine), there is a slight pinkish hue to the solution above the precipitate. In tube 4 from the left (methylene blue), there is a slight bluish hue to the solution above the precipitate.

From this experiment, it is clear that all model dye compounds were strongly assimilated by crystalline cellulose gel, as evidenced by strong coloring of the gel sediment at the bottom of the tubes. However, as signified by liquid phase color change intensity, compound release to the media from the crystalline cellulose gel differed considerably among samples.

The detailed analysis of absorbance in the liquid phase revealed that dye release from crystalline cellulose lattice to the liquid phase differed considerably among compounds. Based on a rudimentary analysis, the tested compounds can be categorized in groups of very slow, slow, fast and very fast release patterns from the crystalline cellulose gel.

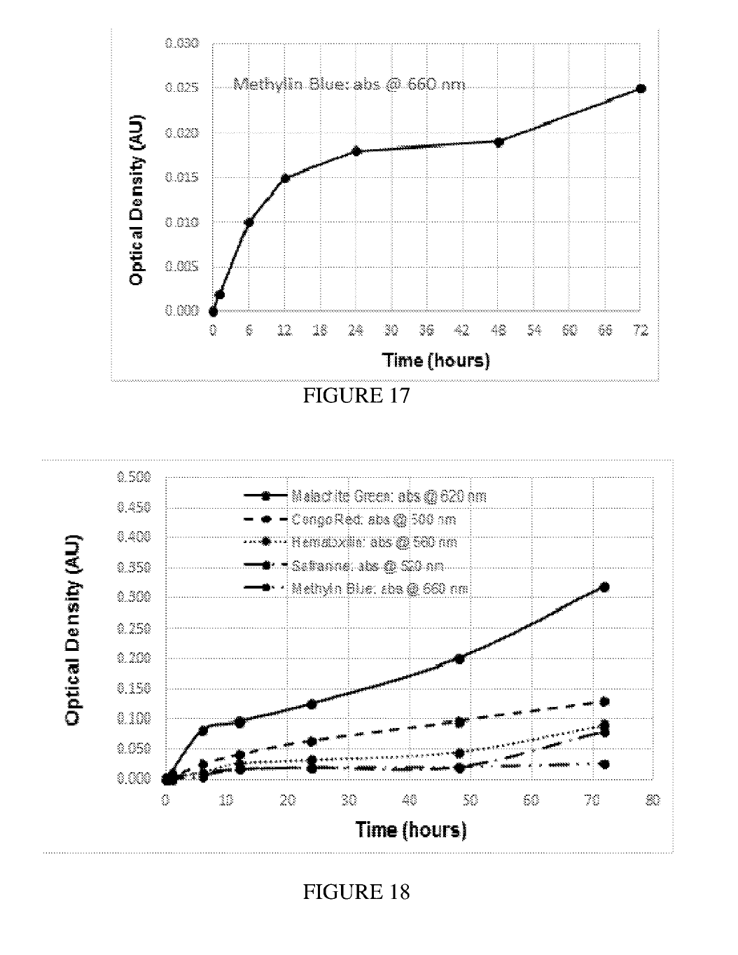

A prominent example of compounds representing a very slow releasing characteristic is methylene blue (FIG. 17). Notably, based on spectrophotometric data, a very small amount of methylene blue (equivalent to 0.025 absorbance units (AU) over 72 hours) was released from the cellulose matrix to the aqueous medium. This indicated that chemical structure of this dye confers very strong affinity with the cellulose lattice.

Examples of compounds representing slow releasing characteristics include malachite green, congo red, haematoxylin, and safranine (FIG. 18). The release profile of methylene blue is included as a point of reference. Notably, dye release in liquid phase equivalent to 0.32, 0.128, 0.09, and 0.08 AU over 72 hours for malachite green, congo red, haematoxylin, and safranine respectively was several fold higher in comparison to methylene blue.

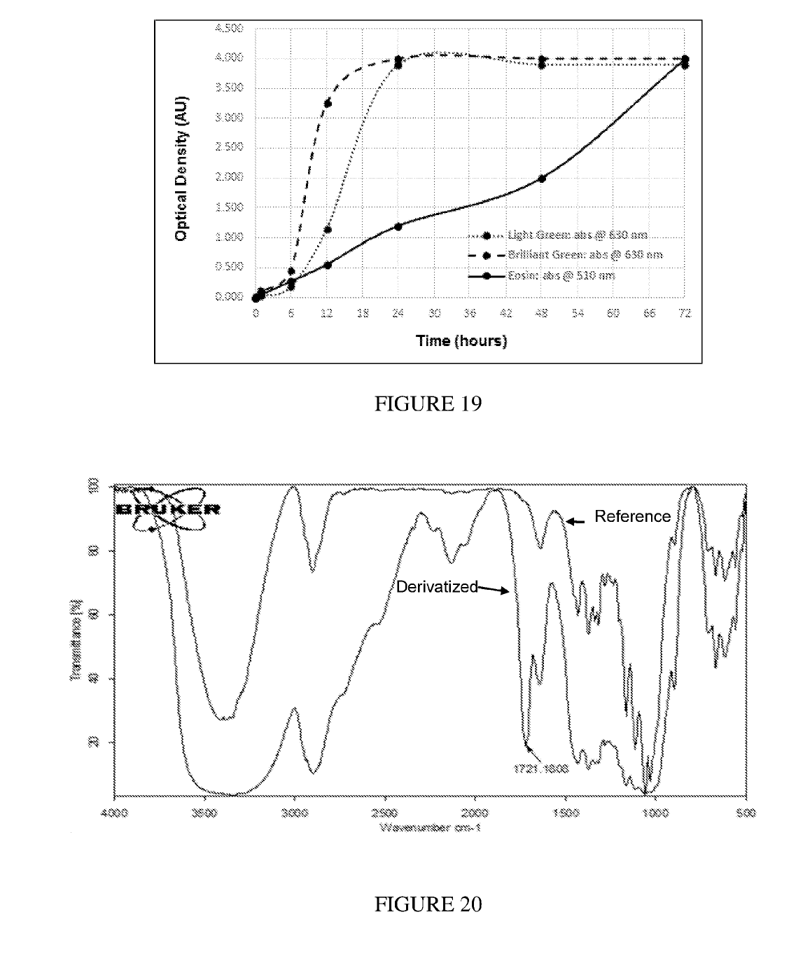

An example of a compound representing fast release is eosin, and compounds exhibiting very fast releasing characteristics include light green and brilliant green (FIG. 19). It is noteworthy that, although the release of all three of these compounds reached saturation (equivalent to 4 AU) at 72 hours in the liquid phase, the pattern of release for eosin differed from the other two compounds.

Without being bound by theory, it is believed that the differences in the release profiles observed for the tested exemplary dyes may be a result of the overall net charge (i.e. polarity) of the different dye molecules. Based on these results, it appears that compounds with dominant positive polarity are slowly released, whereas compounds with dominant negative polarity are released quickly. However, other molecular characteristics such non polar moieties, and relative distribution of polar elements may influence the interactions of any given compound with cellulose lattice, and thus may also affect assimilation and release dynamics.

This experiment also demonstrates that crystalline cellulose gel is able to absorb and release aromatic compounds having a highly complex phenolic ring structure that is common among many dyes and pharmaceutical agents, suggesting crystalline cellulose gel will be useful for applications such as drug delivery.

### Example 9.0

### Surface Modification of Crystalline Cellulose

The inventors tested carboxylation as a desirable functional surface modification of crystalline cellulose gels. Other forms of modification that could be carried out include esterification, etherification, cationisation, silylation, amidation, polymer grafting, and others, and it would be within the expected ability of a person of ordinary skill in the art to carry out suitable surface modification of the cellulose moieties of the crystalline cellulose gel based on a desired application or desired properties of the crystalline cellulose gel. One rationale for selecting carboxylation is that most of the known poorly soluble or insoluble drugs are basic, and providing carboxylic groups may facilitate interactions with such basic molecules.

Following a series of experiments, it was found that reacting crystalline cellulose gel with performic acid was a good method for cellulose modification, resulting in an abundance of carbonyl groups. For this purpose a new approach for generation of performate using glycerol as catalyst was also developed. This new procedure replaced a procedure for synthesis of performate which was dependent on using phenol as a catalyst, so the complete process used to carboxylate the crystalline cellulose gel is consistent with green chemistry in its entirety.

The modification of the surface chemistry of crystalline cellulose developed involves three steps: 1) preparation of performate reagent, 2) reaction of crystalline cellulose with performate reagent, and 3) purification of reacted crystalline cellulose derivatives.

The performate reagent is prepared by reacting 10 ml of hydrogen peroxide (35%) and 90 ml of formic acid (88%). Any suitable volume ratio can be used; in this example, the volume ratio used is 10:90. Other ratios can also be used to generate performate, but the use of a very high ratio of hydrogen peroxide may result in a risk of explosion upon heating. In order to increase the efficiency of performate synthesis a catalyst is added to reaction. Prior processes used 500 mg of phenol/100 ml of reaction mixture. This process was modified by the inventors so that the phenol is replaced by 5 ml of glycerol (100%). Any suitable range of glycerol can be used, for example between 1% to 5% vol./vol., or any value therebetween, e.g. 2%, 3% or 4% vol./vol. The reaction is allowed to develop at room temperature for at least 1 hour. The reaction is exothermic, producing heat.

For the surface modification process, crystalline cellulose gel is mixed with performate reagent to form approximately a 10% vol./vol. slurry of performate reagent relative to the volume of crystalline cellulose gel present. Any suitable concentration of performate reagent can be used, and the relative amount of performate reagent used can be increased if desired. The mixture is allowed to react for several hours at room temperature (e.g. more than five hours, usually overnight). The reaction is exothermic, producing heat. At the completion, the reacted cellulose is separated by filtration and washed in reverse osmosis water to obtain a neutral suspension.

This process is very effective for the generation of carbonyl groups as evidenced by FTIR spectroscopy (FIG. 20). Noteworthy is a prominent deflection on the lower tracing (representing the derivatized carboxylated product) at 1721 cm⁻¹ wave number, which is indicative of a functional carbonyl group on the glucose molecule. The upper tracing is the reference native crystalline cellulose, and does not have this deflection.

An important aim of chemical functionalization is the introduction of stable electrostatic charges on the surface of cellulose crystals. It may be possible to manipulate the secondary structure of crystalline cellulose during the process of synthesis to enhance its custom designed physico-chemical environment. For instance, modification of crystalline cellulose glucose moieties by adding anions such as carboxylic functional groups would provide a 3-dimensional interior environment consistent with a cryptand as a binding site (a nook) for “guest” molecules, including ions (for example, drugs or other biologically useful molecules).

### Example 10.0

### Functional Study—Interaction of Crystalline Cellulose Gel-Stabilized Emulsions with White Blood Cells

In order to evaluate the putative viable cell-vesicle interactions, an assay was developed in which freshly isolated viable white blood cells were allowed to interact with crystalline cellulose-based vesicles.

White blood cells were isolated from whole blood according to the procedure described previously (Carlson, G. P., and J. J. Kaneko, 1973: Isolation of leukocytes from bovine peripheral blood. Proc. Soc. Exp. Biol. Med. 142, 853-860) with some modification. Briefly: blood samples were collected from the vein into vacutainers containing EDTA for the preparation of white blood cells (WBC) and into plain vacutainers for the separation of serum. First, whole blood was centrifuged at 2000 g for 10 minutes to separate packed cell volume and plasma. Plasma was decanted, and two ml of blood cells were transferred to a glass tube followed by the addition of 4 ml of deionized water. After gentle mixing for 30 seconds, the isotonicity was restored by the addition of 2 ml phosphate buffered saline (PBS) containing 450 mmol L⁻¹ sodium chloride (NaCl). The tubes were next centrifuged at 1500 g for 10 minutes. The red blood cell (RBC) lysate was discarded and the sediment containing white blood cells was resuspended in 4 ml of deionized water, gently mixed for 15 seconds and the isotonicity restored as described earlier. The tubes were next centrifuged at 1500 g for 5 minutes and the pellet washed twice with isotonic PBS containing 150 mmol L⁻¹ NaCl. The resultant pellet containing white blood cells was re-suspended in Hank's solution (HBSS, pH 7.4). The entire procedure was carried out at room temperature (23° C.). The isolated cells were counted in a haemocytometer and the viability determined by dye exclusion using 2×10⁻¹ mmol L⁻¹ methylene blue. The white blood cell preparation routinely contained a minimum of 90% of viable white blood cells (predominantly neutrophils).

For the purpose of the assay, the vesicles were prepared using flax crystalline cellulose gel (approximately 1% w/v in reverse osmosis water) combined with flax oil as describe above in Example 6.0. A sufficient amount of flax oil was added to provide a saturated emulsion, with no unencapsulated oil present in the emulsion. For the purpose of the assay using viable cells, the final preparation of vesicles from cellulose gel/flax oil emulsion was re-suspended in isotonic PBS solution, and the pH was adjusted to 7.4 with dibasic potassium phosphate.

The evaluation of interactions between white blood cells and vesicles was carried out using native and opsonized vesicles. Freshly prepared homologous serum was used for opsonisation. Approximately 25 microliters of serum was mixed with 75 microliters of emulsion in order to allow serum opsonins to bind vesicles. Opsonins bound to target molecules can facilitate phagocytosis by rendering the target molecules immune-reactive, thus making it easier for white blood cells to carry out phagocytosis.

Aliquots of 0.2 ml of suspension containing white blood cells were placed in test tubes, and 50 μL of either native or opsonised vesicles preparation was added to the assay medium. The tubes were capped and incubated for 120 minutes at 39° C. Following this, neutrophil cultures were assessed for the presence of white blood cells with internalized vesicles. The number of cells containing internalised vesicles expressed as % represented an index of cell-vesicle interaction.

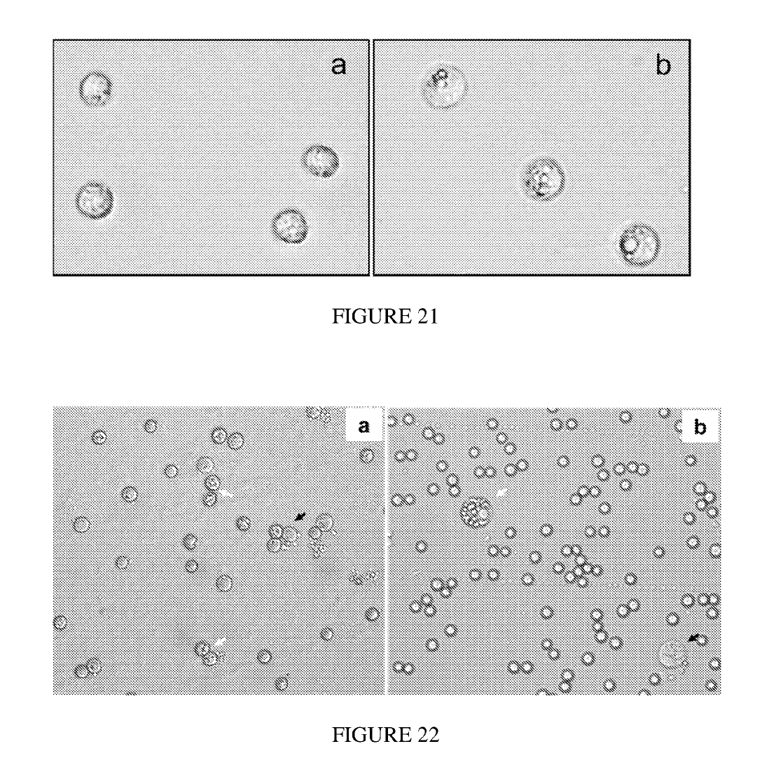

In the present study the interactions of vesicles with blood cells isolated from cattle, horse, and dog were examined. Each assay was replicated at least three times. In all assays, there was clear evidence that white blood cells commenced internalization of vesicles within minutes after exposure. The vesicles internalized within cells were clearly seen on light microscopic examination (FIG. 21). The white blood cells (mostly neutrophils) in control cultures (FIG. 21, panel a) present typical morphology of granulocytes with prominent granules dispersed in the cytoplasm, whereas in the treated cultures (FIG. 21, panel b) the presence of vesicles within the cell body is clearly discernible. Notably, at the completion of incubation (120 min) 85+% of cells contained vesicles. It is also noteworthy that the process of internalization was the same in both native and opsonized assays.

Taken together, the observations from this study indicate that the interactions of crystalline cellulose stabilized vesicles with cells comes naturally, and the process appears to be very efficient. Of note, because the uptake of vesicles was not dependent on opsonisation, the mechanism of internalization cannot be attributed to phagocytosis. Therefore, without being bound by theory other possible mechanisms of vesicle internalization may be utilized, including: 1) direct fusion of the vesicle lipid layer with the cell lipid bi-layer, and/or 2) pinocytosis, where the cell actively engulfs the vesicle. In alternative embodiments, if a vesicle is coated with antigens, uptake by phagocytosis where the cell internalizes a vesicle into a phagosome could be possible.

The foregoing results demonstrate that crystalline cellulose gel-based vesicles can act as micro- or nano-sized delivery vehicles, and can be predicted to facilitate uptake of encapsulated material by cells. Based on the observations from experiments using white blood cells, it can be soundly predicted that white blood cells can be effectively used as delivery vehicles for molecules encapsulated in vesicles stabilized by crystalline cellulose gel, since white blood cells themselves may target specific types of cells.

In one example embodiment, in the situation of tissue infection, the phagocytic cells (e.g. neutrophils) can carry a drug encapsulated in a vesicle stabilized by crystalline cellulose gel to specific target tissue cells because these cells already possess very strong attraction to the tissue or organs affected by inflammatory responses via chemotaxis. In alternative example embodiments, vesicles can be coated with specific proteins which are recognized only by specific cells, and highly specific delivery vehicles can thereby be engineered. Such embodiments might have particular application in cancer or gene therapy, where a specific drug payload (e.g. anti-cancer compound or nucleic acid such as DNA or RNA) can be delivered to a target location in the body with minimal systemic complications.

### Example 11.0

### Functional Study Using Whole Blood

Further trials were designed to evaluate vesicle behavior and interactions with viable cells in the environment of the blood plasma matrix. Vesicles were added to freshly collected blood, and gently mixed. The emulsion stabilized by crystalline cellulose gel was prepared as described above. Blood was collected from the vein into vacutainers containing EDTA, and then transferred in aliquots of 2 ml to two glass test tubes. The blood remaining in the vacutainer was centrifuged at 3000×g to separate blood plasma from blood cells. This procedure was carried out in order to assess the sample for any signs of haemolysis. The plasma separated from packed cell volume (PVC) appeared clear and no evidence of haemolysis was noted.

For the assay, 2 ml blood samples were gently mixed with 200 μL of either PBS (control) or vesicles preparation (treatment), and left undisturbed on the bench. After 30 minutes the assays were gently mixed and a drop of blood from each tube was placed on glass slide and covered with a cover slip for microscopic examination. The tubes were closed with plastic stoppers and the assays were incubated undisturbed on the bench at room temperature for 24 hours.

Following the incubation, the assays were gently agitated to disperse separated packed cell volume (PVC), and drop of blood was from each tube was placed on glass slide and covered with covering slip. The specimens were examined under the light microscope. The blood remaining in the assays was centrifuged at 3000×g, and separated plasma was evaluated for any signs of haemolysis.

The interactions of vesicles with blood cells were apparent within 30 minutes after administration of the treatment, and the internalized vesicles within cells were clearly seen on light microscopic examination (FIG. 22). The plasma separated from packed cell volume (PVC) was clear. Although both control and treatment assays showed very faint reddish coloration indicative that small number red blood cells were haemolysed over 24 hours of incubation, there were no differences between control and treated samples. It was concluded that there were no apparent signs of any haemolysis associated with the treatment.

This assay conducted using whole blood showed that blood cells can interact with vesicles in the environment of blood plasma matrix very effectively, supporting the utility of vesicles stabilized with crystalline cellulose gels in drug delivery applications, including facilitating drug delivery in the blood. Furthermore, this experiment provides evidence that mixing of blood with vesicles does not appear to induce hemolysis. Drug induced hemolytic anemia is a relatively rare but unpredictable complication of drug therapy, and the list of implicated medications is constantly growing.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of this specification as a whole.

1. An emulsion comprising:
an aqueous phase;
an oily phase; and
a crystalline cellulose gel;
wherein the emulsion is stabilized by the crystalline cellulose gel.

2. An emulsion as defined in claim 1, comprising:
between 45% and 98% v/v aqueous phase;
between 1% and 50% v/v oily phase; and
between 0.1% and 5% w/v crystalline cellulose gel.

3. An emulsion as defined in claim 1, wherein the oily phase comprises a plant-based oil, wherein the plant-based oil optionally comprises flax oil, hemp oil, canola oil, corn oil, sunflower oil, safflower oil, soy oil, coconut oil, palm oil, sesame oil, or olive oil; and/or wherein the oily phase comprises an animal-based oil, wherein the animal-based oil optionally comprises fish oil or milk fat; and/or wherein the oily phase comprises a combination of two or more of the foregoing oils.

4. An emulsion as defined in claim 1, wherein the crystalline cellulose gel is obtained from agricultural biomass or forestry by-products, or from bacterial cellulose.

5. An emulsion as defined in claim 4, wherein the agricultural biomass or forestry by-products comprise wood, cotton, cereal straw, grass straw, flax straw, or hemp straw; wherein the cereal straw optionally comprises wheat, oat, barley or rice straw; and wherein the flax straw or hemp straw optionally comprises shives (hurd), bast fiber, or whole straw.

6. (canceled)

7. An emulsion as defined in claim 1, comprising an active agent or biological molecule.

8. An emulsion as defined in claim 7, wherein the active agent or biological molecule comprises a drug, nutrient, vitamin, mineral, amino acid, prebiotic, carbohydrate, nucleic acid including deoxyribonucleic acid (DNA) or ribonucleic acid (RNA), dye, hormone, or functional food ingredient, wherein the functional food ingredient optionally comprises a fatty acid, conjugated linoleic acid, carbohydrate, or an anti-oxidant; or wherein the biological molecule comprises an aromatic compound, phenolic rings, secondary, tertiary or quaternary nitrogen, an imidazole ring, or an antifungal drug.

9. An emulsion as defined in claim 8, wherein the active agent or biological molecule is encapsulated within the dispersed phase.

10. An emulsion as defined in claim 1, wherein the emulsion comprises an oil-in-water emulsion.

11. An emulsion as defined in claim 1, wherein the crystalline cellulose gel is obtained by breakdown of cellulose obtained from biomass by a catalytic reaction using an iron-based nanoparticulate catalyst in acidic solution containing hydrogen peroxide.

12. An emulsion as defined in claim 1, wherein the emulsion comprises unilamellar vesicles and/or multilamellar vesicles.

13. An emulsion as defined in claim 1, wherein the emulsion comprises a nano-emulsion or a micro-emulsion.

14. A vesicle derived from an emulsion as defined in claim 1, wherein the vesicle is optionally a nanoparticle sized vesicle.

15. A method of producing an emulsion, the method comprising:
combining an aqueous phase and an oily phase with a crystalline cellulose gel; and
mixing to produce an emulsion.

16. A method as defined in claim 15, wherein mixing to produce an emulsion comprises shaking, stirring, vortexing, homogenizing, fluidizing, cavitating, or sonicating.

17. (canceled)

18. The method as defined in claim 15, further comprising a step of using the emulsion to:
encapsulate a nutrient, including a mineral, vitamin, functional food ingredient, amino acid, or a prebiotic, for inclusion in a food or feed product, wherein the food or feed product is optionally a nutritional supplement or a low calorie food product, and wherein the functional food ingredient optionally comprises a fatty acid, conjugated linoleic acid, carbohydrate, or an anti-oxidant;
enhance the bioavailability of a nutrient, including a mineral or vitamin, functional food ingredient, or amino acid, or a prebiotic, in a food or feed product, wherein the food or feed product is optionally a nutritional supplement or a low calorie food product, wherein the functional food ingredient optionally comprises a fatty acid, conjugated linoleic acid, carbohydrate, or an anti-oxidant;
encapsulate a prebiotic, an oil, or a nutrient, including a mineral or vitamin, functional food ingredient, or amino acid, and prepare a suspension, cream, paste or foam for inclusion in a food or feed product, wherein the food or feed product is optionally a nutritional supplement or a low calorie food product, wherein the functional food ingredient optionally comprises a fatty acid, conjugated linoleic acid, carbohydrate or an anti-oxidant;
encapsulate a prebiotic, an oil, or a nutrient, including a mineral or vitamin, functional food ingredient, or amino acid for delivery to the gastrointestinal tract of an animal, optionally for delivery to the small intestine or large intestine of a ruminant, wherein the functional food ingredient optionally comprises a fatty acid, conjugated linoleic acid, carbohydrate, or an anti-oxidant;
form a multilamellar vesicle encapsulating a drug or other bioactive compound to provide time-delayed and/or sustained release of the drug or other bioactive compound in an animal, including a mammal, including a human, wherein the multilamellar vesicle is optionally formulated for administration orally, topically, including as a skin patch, parenterally, transmucosally, by inhalation, or by injection;
form a vesicle encapsulating a drug or other biomolecule including a hormone, dye or a nucleic acid, including deoxyribonucleic acid (DNA) or ribonucleic acid (RNA), to deliver the drug or other biomolecule to cells in the body of an animal, including a mammal, including a human;
form a vesicle encapsulating a drug or other biomolecule including a nucleic acid, including deoxyribonucleic acid (DNA) or ribonucleic acid (RNA), to provide targeted delivery of the drug or other biomolecule to desired cells in the body of an animal, including a mammal, including a human, by modifying the crystalline cellulose gel to attach a targeting moiety that targets the vesicle to desired cells, wherein the targeting moiety comprises a ligand, an opsonin, cell receptor active substances, monoclonal antibodies, or custom-design antigens;
form a pharmaceutical composition incorporated into a formulation, emulsion, suspension, cream, lotion, paste, ointment, foam, or skin patch;
form a pharmaceutical composition suitable for administration orally, topically, parenterally, transmucosally, by inhalation, or by injection;
encapsulate a dye or a protective coating for use in a paint or coating application, wherein the paint or coating is optionally formulated to be applied to a surface such as metal, plastic or wood;
assist in product recovery operations, wastewater treatment, remediation of petroleum contaminants in soil, and/or the manufacture of explosives for mining; or
form a cryptand for encapsulating a guest molecule, wherein the crystalline cellulose gel is optionally modified to provide a desired physico-chemical environment for the guest molecule, for example by cross-linking glucose moieties of the crystalline cellulose gel with cations such as amino (NH₄⁺) functional groups or anions such as carboxylate (COO⁻) functional groups.

19. An emulsion as defined in claim 1, wherein the crystalline cellulose gel comprises native crystalline cellulose.

20. An emulsion as defined in claim 1, wherein the cellulose comprising the crystalline cellulose gel is chemically modified, and wherein the chemical modification optionally comprises esterification, etherification, amidation, cationisation, carboxylation, silylation, and/or polymer grafting.

21. (canceled)

22. (canceled)

23. An emulsion as defined in claim 1, wherein the crystalline cellulose gel comprises thixotropic properties, wherein the crystalline cellulose gel comprises a hydrogel, wherein the crystalline cellulose gel exhibits dichroism, and/or wherein the crystalline cellulose gel cannot be resuspended back into a gel after the crystalline cellulose gel has been dried.

24. A method of using an emulsion prepared by the method of claim 15 to deliver a drug or other biological molecule to blood cells of an animal, wherein the blood cells optionally comprise white blood cells of the animal, or wherein the drug optionally comprises an anti-malaria drug and the blood cells comprise red blood cells of the animal.

25. (canceled)

26. (canceled)
